(19) United States
(12) Patent Application Publication
(10) Pub. No.: US 2009/0253620 A1
(43) Pub. Date: Oct. 8, 2009

(54) METHODS AND COMPOSITIONS FOR TREATING GASTROINTESTINAL RADIOSENSITITIVITY IN A SUBJECT

(75) Inventors: Jeffrey Gordon, St. Louis, MO (US); Peter Crawford, University City, MO (US)

Correspondence Address: POLSINELLI SHUGHART PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 12/063,783

(22) PCT Filed: Aug. 18, 2006
(86) PCT No.: PCT/US06/32423

§ 371 Date: May 29, 2008

Related U.S. Application Data
(60) Provisional application No. 60/709,916, filed on Aug. 19, 2005.

(51) Int. Cl. A61K 38/16; A61K 31/7088

(52) U.S. Cl. 514/12; 514/44 R

(57) ABSTRACT

A method and a related composition for treating gastrointestinal radiation sensitivity in a subject are disclosed. The method comprises administering to the subject an agent that increases either the amount of or the activity of a fasting-induced adipose factor (Fiaf) protein in a subject. The composition comprises a first agent that increases either the amount of or the activity of a fasting-induced adipose factor (Fiaf) protein in a subject and a second agent that ameliorates at least one symptom of gastrointestinal radiosensitivity in a subject.

### GOVERNMENTAL RIGHTS IN INVENTION

This work was supported by the U.S. Department of Health and Human Services/National Institutes of Health grant number DK30292. The U.S. Government has certain rights in this invention.

### FIELD OF THE INVENTION

The present invention generally relates to treating gastrointestinal radiation sensitivity in a subject. More specifically, the present invention relates to reducing the incidence of cellular apoptosis in the gastrointestinal tract of a subject, thereby treating gastrointestinal radiosensitivity. The invention provides methods and compositions to reduce gastrointestinal radiosensitivity in a subject by increasing either the amount or the activity of the fasting-induced adipose factor (Fiaf) polypeptide in the subject.

### BACKGROUND OF THE INVENTION

### Fiaf Induction

The mammalian gastrointestinal tract is inhabited by >500 species of microorganisms, most of which are bacteria. This ‘bacterial nation’ (microbiota) outnumbers our somatic and germ cells by at least ten fold, and its collective genome contains ˜100-fold more genes than ours. The microbiota is an important regulator of mammalian nutrition, gut motility, intestinal epithelial cell turnover, mucosal barrier functions, and immune function. Studies of mice reared in the absence of a microbiota (germ-free; GF) and those that have acquired a microbiota from the time of birth have disclosed numerous ways in which indigenous microbes affect the host. Particularly pertinent to the present application is the observation that germ-free (GF) mice are less susceptible to gastrointestinal radiation sensitivity than mice with a microbiota.

### Radiation Therapy

The use of radiation therapy (also referred to as radiotherapy, x-ray therapy, or irradiation) to treat cancer is well known in the art. At least half of all patients treated for cancer (U.S. incidence 1.3 million in 2002, National Cancer Institute data) receive radiation therapy. Radiation therapy may be external, internal, or systemic. Internal radiation therapy typically comprises an implant, containing the source of radiation, which is inserted in a tumor or in close proximity to a tumor. The most common type of radiation therapy is external, where the radiation is generated by a machine external to the body. The source of radiation used in therapy may be either electromagnetic or particle radiation. Examples of suitable sources of radiation include alpha particles, beta particles, gamma rays (electromagnetic), X-rays (electromagnetic), and ultra violet radiation. More specifically, numerous radioactive isotopes are suitable sources of radiation—alone or in combination with other isotopes, including, for example, cesium-137 or cobalt-60. Different types of radiation therapy may be combined and radiation therapy may be used alone or in combination with other cancer treatments, such as chemotherapy or surgery.

The type and exact dose of radiation used in a treatment program varies with such factors as the species, age, sex and physical condition of the subject, the subject's overall treatment plan (i.e., whether other types of treatment are being used), and the location and nature of the cancer involved.

The use of radiation to treat cancer is derived from the fact that cancer cells proliferate and have a diminished ability to repair sub-lethal damage compared to most healthy differentiated cells. Radiation therapy affects cells by damaging their genetic material. Because DNA damage is inherited through cell division, rapidly dividing cells accumulate damage and die or reproduce more slowly. Cancer cells as well as certain healthy cells, such as endothelial cells of the intestinal capillaries and epithelial stem cells located at the bases of intestinal crypts, are particularly sensitive to radiation. The administration of radiotherapy is driven and limited by the balance of intended destruction of neoplastic cells and undesired damage to normal surrounding tissue.

The radiosensitive nature of endothelial cells of the intestinal capillaries and intestinal epithelial stem cells is believed to contribute to gastrointestinal radiation sensitivity and radiation enteritis, in subjects undergoing radiotherapy to the abdomen, intestine, pelvis, or total body. Radiation enteritis limits the dose of radiation that can be administered for abdominal and pelvic malignancies as well as the dose that can be used for total body irradiation. It is not fully understood how radiation specifically triggers these conditions, but there are several possible explanations. Radiation may cause these conditions by injuring epithelial stem cells and villi. Damage to epithelial stem cells eventually leads to the deterioration of the mucosal barrier in the intestine, which subsequently leads to various disorders, such as diarrhea, proctitis, malnutrition, and systemic infection. More recently, it has been reported that radiation affects and injures endothelial cells of the intestinal capillaries. A mechanism underlying radiation enteritis is cellular apoptosis in the endothelial cells of intestinal capillaries. Regardless of the exact mechanism, the increased incidence of cellular apoptosis due to exposure of cells to radiation contributes greatly to gastrointestinal radiation sensitivity, radiation enteritis, and the various related disorders, including diarrhea, dehydration, malnutrition, weight loss, and systemic infection.

Current treatment options for gastrointestinal radiation sensitivity, particularly enteritis, by and large are directed toward treating these related disorders. The National Cancer Institute recommends, as a primary treatment for enteritis, treating the diarrhea, loss of fluids, poor absorption, and stomach or rectal pain associated with enteritis. The Institute advises treating these conditions with medications, i.e., antidiarrheal medications, changes in diet, and rest, Tellingly, the National Cancer Institute advises that if such conditions worsen, even with the recommended treatment, then cancer treatment may have to be discontinued, at least temporarily.

Gastrointestinal radiation sensitivity is a dose-limiting and life-threatening complication for both region-focused γ-irradiation for pelvic and abdominal malignancies, as well as total body irradiation for bone marrow transplantation. There is a need for a therapeutic agent capable of reducing gastrointestinal radiosensitivity, particularly radiation enteritis, and increasing endothelial survival.

### SUMMARY OF THE INVENTION

Among the several aspects of the invention is provided a method for treating gastrointestinal radiation sensitivity in a subject, the method comprising administering to the subject an agent that increases either the amount or the activity of a Fiaf polypeptide in the subject.

Another aspect of the invention provides a method for reducing the incidence of cellular apoptosis in the gastrointestinal tract of a subject, the method comprising administering to the subject an agent that increases either the amount or the activity of a Fiaf polypeptide in the subject.

A further aspect of the invention provides a composition for decreasing gastrointestinal radiosensitivity in a subject. Generally, the composition comprises a first agent that increases either the amount or the activity of a Fiaf polypeptide and a second agent that ameliorates at least one symptom of gastrointestinal radiosensitivity.

Other aspects and features of the invention will be in part apparent and in part pointed out hereinafter.

### BRIEF DESCRIPTION OF THE FIGURES

FIG. 1 depicts a series of images illustrating microbiota-associated radiosensitivity of small intestinal villus core mesenchymal cells to total body irradiation (TBI)-induced apoptosis. TUNEL⁺ apoptotic cells appearing 4 h after 16 Gy TBI in the distal small intestines of conventionally-raised (CONV-R) (FIG. 1A), conventionalized (CONV-D) (FIG. 1B), and germ-free (GF) (FIG. 1C) wt FVB/N mice. Fluorescence microscopy reveals green TUNEL⁺ cells in the crypt epithelium (arrowheads) of all three mice and in the villus core mesenchyme (arrows) of CONV-R and CONV-D mice. In contrast, TUNEL⁺ cells are absent from the villus core mesenchyme of the GF animal. Scale bars are equal to 50 μm. FIG. 1D is a graph depicting the mean number (±1 S.D.) of TUNEL⁺/DAPI⁺ mesenchymal cells per distal small intestinal villus sections prepared from GF, CONV-R, CONV-D, and B. theta/E. coli bi-associated FVB/N mice (4-5 mice scored/group; ≧100 villus sections assayed/animal). ***, p<0.001 compared to GF. In non-irradiated CONV-R controls, there were ≧0.2 apoptotic cells per section of villus core mesenchyme.

FIG. 2 depicts a series of images illustrating apoptosis of CD31⁺ endothelial cells and CD45⁺ leukocytes in the villus mesenchyme of a CONV-R mouse treated with 16 Gy TBI. FIG. 2A depicts the small intestinal villus section stained with hematoxylin and eosin (left panel; Scale bar, 100 μm). The expanded views of the boxed region in the right panels show an adjacent section after staining with TUNEL reagents and antibodies to CD31. The arrow points to a TUNEL⁺ nucleus in a CD31-positive endothelial cell. Scale bar is equal to 20 μm. FIG. 2B is a transmission electron microscopic (TEM) image of a mesenchymal endothelial cell (arrowhead) from the distal small intestine of a CONV-R mouse 4 h after 16 Gy TBI. Nuclear condensation and cellular protrusion into the capillary lumen are characteristics of endothelial apoptosis. Scale bar is equal to 2 μm. FIG. 2C is a TEM image of a representative villus mesenchymal endothelial cell (arrowhead) from the distal small intestine of a GF C57Bl/6 mouse killed 4 h after 16 Gy TBI. The cell has normal morphology. Abbreviations: Epi, epithelium; Mes, mesenchyme, bb, brush border on the apical surface of enterocytes. Scale bar is equal to 4 μm. FIG. 2D is a single image from 3D reconstruction of serial 1-μm thick scans of a villus section from a CONV-R mouse killed 4 h after 16 Gy TBI. Arrows, TUNEL⁺CD45⁺ cells; arrowheads, TUNEL⁺CD45⁻ cells. CD45 is a surface marker, while TUNEL stains nuclei: therefore, TUNEL⁺ cells that co-express CD45 will not appear yellow. The insert in the lower right corner is a higher power view of the boxed area. Scale bar is equal to 80 μm.

FIG. 3 depicts a series of images illustrating, through the use of Rag1⁻/⁻ mice, that the microbiota-dependent apoptotic response of villus endothelial cells to 16 Gy TBI does not require mature T-cells or B-cells. FIG. 3A depicts a quantitative assessment of TUNEL⁺DAPI⁺ cells in the villus mesenchyme of B6 GF and CONV-D wt and Rag1⁻/⁻ mice sacrificed 4 h after 16 Gy TBI (n=4-6 mice/group; ≧100 villus sections assayed per mouse). ***, p<0.001 compared to GF Rag1⁺/⁺; **, p<0.01 compared to GF Rag1⁻/⁻ mice; Ψ, p<0.05 compared to CONV-D Rag1⁺/⁺ mice. FIG. 3B depicts two TUNEL⁺ CD31⁺ endothelial cells (arrows) in the small intestinal villus mesenchyme of a CONV-D Rag1⁻/⁻ mouse treated as in panel A. The image was acquired from a 0.5 μm-thick digital scan of the section. FIG. 3C depicts an adjacent section showing two TUNEL⁺CD45⁺ leukocytes (arrows) in the villus mesenchyme. FIG. 3D depicts microscopic angiograms showing single images from 3D reconstructions of serial 1 μm-thick scans of non-irradiated distal small intestinal villi from CONV-D Rag1⁺/⁺ and Rag1⁻/⁻ distal small intestinal villi. Scale bars are equal to 20 μm.

FIG. 4 depicts a series of graphs and images illustrating that loss of Fiaf results in loss of resistance to TBI-induced apoptosis in GF mice. FIG. 4A is a graph showing the results of a quantitative real time polymerase chain reaction (qRT-PCR) study. qRT-PCR analysis shows that conventionalization of GF mice suppresses Fiaf expression in the distal small intestine and that this effect is not abrogated following 16 Gy of TBI (mean±1 S.D.; n=4 mice/group; *, p<0.05, compared to GF untreated B6 mice; **, p<0.01 compared to GF mice treated with 16 Gy TBI 4 h prior to sacrifice). FIG. 4B depicts a graph showing quantitative assessment of apoptosis in the villus mesenchyme of GF Fiaf⁻/⁻ mice and their wt littermates 4 h after 18 Gy TBI (n=9 mice/group; ≧100 villus sections assayed per mouse; mean±1 S.D.)**, p<0.01 compared to GF Fiaf⁺/⁺. FIG. 4C depicts single images from digital 3D reconstructions of small intestinal villus capillary networks in GF Fiaf⁻/⁻ and wt littermates. Scale bars are equal to 20 μm. FIG. 4D depicts an image of TUNEL⁺CD31⁺ endothelial cell (arrow) in the small intestinal villus mesenchyme of a GF Fiaf⁻/⁻ mouse treated as in panel A. Scale bar is equal to 50 μm. FIG. 4E is a TEM image of a villus mesenchymal endothelial cell in a Fiaf⁻/⁻ mouse sacrificed 4 h after 18 Gy TBI. Note the nuclear protrusion and avulsion from the basement membrane (arrow), two ultrastructural manifestations of endothelial cell death. Scale bar is equal to 5 μm. FIG. 4F is an image of TUNEL⁺CD45⁺ leukocyte (arrow) in the small intestinal villus mesenchyme of a GF Fiaf−/− mouse treated as in panel A. Scale bar is 20 μm.

FIG. 5 is a series of images illustrating that GF mice are resistant to lethal radiation enteritis. FIG. 5A depicts survival curves for wt GF, CONV-R, and CONV-D mice after receiving 16 Gy TBI followed by bone marrow transplantation (BMT) from syngeneic donors. **, p<0.005 compared to GF; ***, p<0.001 compared to GF. FIG. 5B depicts an image of hematoxylin and eosin-stained section from the distal small intestine of a peri-mortem GF mouse sacrificed 10 d after 16 Gy TBI (the mouse did not receive a BMT and therefore died of hematopoietic failure). There is no evidence of radiation enteritis. FIG. 5C depicts an image of hematoxylin and eosin-stained section from the distal small intestine of a CONV-R mouse treated with 16 Gy TBI followed by BMT. The mouse was sacrificed as soon as it became moribund, 5 d after treatment. Features of radiation enteritis are evident: mucosal atrophy; mesenchymal inflammation and fibrosis. Inset shows the distal intestine from a non-irradiated wt CONV-R control. Scale bars are equal to 50 μm.

FIG. 6 depicts a series of images illustrating that the villus mesenchyme is populated by leukocytic but not endothelial descendants of transplanted bone marrow progenitors. FIG. 6A depicts results of a multi-label immunohistochemical study showing GFP-expressing endothelial cells in the small intestinal villus core mesenchyme of a CONV-R B6 marrow donor harboring a knock-in of the EGFP gene into the Sca1 locus. Arrows point to GFP⁺CD31⁺ cells. Images were acquired by serial digital sectioning (section plane thickness=0.5 μm). Scale bar is 20 μm. FIG. 6B illustrates results from a recipient wt B6 mouse 12 weeks after receiving 10 Gy TBI and BMT with Sca-1-GFP⁺ donor marrow. Despite the presence of numerous GFP⁺ endothelial cells in the Sca1-EGFP donor (6A), donor-derived GFP-expressing cells are not incorporated into the CD31⁺ endothelium after TBI. FIG. 6C depicts evidence demonstrating that donor-derived GFP⁺ cells differentiate into leukocytes within the villus mesenchyme of BMT recipients. Adjacent section from same recipient mouse as in 6B. Arrows point to CD45⁺GFP⁺ donor-derived cells. Inset: same villus section, without GFP channel, Scale bars are equal to 50 μm.

### DETAILED DESCRIPTION OF THE PREFERRED EMBODIMENTS

The present invention provides methods for treating radiosensitivity in a subject. Typically the method involves administering an agent that increases either the amount or activity of a Fiaf polypeptide. Additionally, the method may be utilized to reduce the incidence of cellular apoptosis in the gastrointestinal tract of a subject. The invention encompasses compositions as well as methods of treatment. Advantageously, the treatments of the present invention limit morbidity and mortality following abdominal, pelvic, or total body irradiation, allow for an increased dose of radiotherapy to be administered, and allow for enhanced endothelial survival and function.

### I. Gastrointestinal Radiation Sensitivity

Radiation therapy exerts its effects on rapidly dividing cells, such as cancer cells or the cells lining the large and small intestine, as well as endothelial cells. With respect to cancer cells, this is the intended effect of radiation therapy. In contrast, with respect to the non-cancerous cells of the gastrointestinal system, this effect may result in radiation sensitivity, an unwanted side effect. The present invention provides compositions and methods that may be employed to treat gastrointestinal radiosensitivity in subjects undergoing a variety of different radiation treatment regimes. In certain embodiments, the subject may have symptoms of gastrointestinal radiosensitivity, such as a radiation enteritis disorder. In other aspects of the invention, the subject may be at risk for developing gastrointestinal radiosensitivity, i.e. the subject may soon undergo radiation treatment for a gastrointestinal cancer. Irrespective of the embodiment, the method of the invention involves administering to the subject an agent that increases the amount or activity of a Fiaf polypeptide, as described below.

Generally speaking, the subject is a vertebrate. In one embodiment, the subject may be mammalian. In another embodiment, the subject may be a fish. In yet another embodiment, the subject may be a bird. In still another embodiment, the subject may be selected from the group comprising a human, a rodent, a companion animal, or a farm animal. Non-limiting examples of rodents include a mouse, a rat, or a guinea pig. Non-limiting examples of companion animals include a dog, a cat, a rabbit, or a bird. Non-limiting examples of farm animals include a pig, a cow, a horse, a sheep, a chicken, or a goat. In some embodiments, the subject is human. In other embodiments, the subject is a rodent.

In one typical aspect of the invention, the subject is administered radiation to the abdominal or pelvic region of the subject's body. In a further embodiment, the subject may be exposed to total body irradiation. Gastrointestinal radiation sensitivity is implicated in a radiation therapy that exposes a part of the gastrointestinal system to a dose of radiation. The dose of radiation, depending on the subject and the fractionation regimen, for example, may be from about 1 to about 90 Gy. In particular, gastrointestinal radiation sensitivity may develop in a subject undergoing radiation therapy that exposes the abdomen, pelvis, rectum, intestine, or total body to a dose of radiation, such as γ-irradiation. In one alternative of this embodiment, the subject will be undergoing total body irradiation in preparation for bone marrow transplantation. In another alternative of this embodiment, the subject will be undergoing treatment for cancer that includes radiation therapy. Generally speaking, gastrointestinal radiation sensitivity may occur as a result of radiation treatment of a variety of cancers. Non-limiting examples include rectal cancer, cancer of the anus, anal canal and anorectum cancer, peritoneum, omentum and mesentery cancer, cervical cancer, uterine cancer, ovarian cancer, colon cancer, cancer of the small intestine, prostate cancer, stomach cancer, cancer of the bladder, testicular cancer, kidney cancer, pancreatic cancer, liver cancer, vulvar cancer, cancer of the gallbladder and biliary tract, ureteral cancer, and gastrointestinal carcinoid tumors.

In addition to treating gastrointestinal radiosensitivity in a subject, the invention provides treatment methods for gastrointestinal injuries related to radiation sensitivity. In one such embodiment, the method of the invention promotes survival of endothelial cells. In yet another embodiment, the invention provides methods for reducing the incidence of cellular apoptosis in the gastrointestinal tract of the subject.

A further aspect of the invention provides methods for treating either acute or chronic radiation enteritis. In general, gastrointestinal radiation sensitivity manifests itself as a condition known as radiation enteritis, also known as radiation enteropathy, gastrointestinal (GI) syndrome, or radiation-induced bowel injury. Radiation enteritis encompasses various disorders, including diarrhea, dehydration, malabsorption, abdominal pain, rectal pain, proctitis, weight loss, nausea, vomiting, fatty stools, bloody stools, and frequent urges to have a bowel movement. Depending upon the degree of severity, there are two types of radiation enteritis, acute radiation enteritis and chronic radiation enteritis. Acute enteritis symptoms are defined as those that develop during the first course of radiation and up until some eight weeks after the last treatment. Acute enteritis that continues months after the last treatment is characterized as chronic enteritis. Chronic enteritis may develop months to years after radiation treatments are completed. In general, several factors dictate the presence and severity of radiation enteritis, including the dose of radiation, the size of the tumor and the occurrence of metastasis, the fraction of the gastrointestinal tract that was exposed to radiation, use of radiation implants, concurrent chemotherapy, and various factors inherent to the subject, i.e., previous abdominal surgery.

A further embodiment encompasses treating a subject in need of treatment for gastrointestinal radiosensitivity. In certain embodiments, for example, the subject may be diagnosed with radiation enteritis, Radiation enteritis is typically diagnosed by performing a physical exam and examining a subject's medical history. As part of the diagnosis, a physician may assess the usual pattern of bowel movements, the pattern and nature of diarrhea, if present, (i.e., frequency or bloody diarrhea), the presence of disorders listed above (i.e., nausea, abdominal cramping, or proctitis), the nutritional status of the subject (i.e., dehydration or malnutrition), and lifestyle patterns. In certain cases, a colonoscopy or upper endoscopy is performed to examine the lining of the intestine. Additionally, biopsies can be performed during a colonoscopy or an endoscopy.

### II. Administration of an Agent to Increase Fiaf Polypeptide or Fiaf Activity

The method of the invention, irrespective of the embodiment or indication treated, typically involves administering an agent that increases either the amount or activity of a Fiaf polypeptide in the subject. In certain embodiments, the Fiaf polypeptide may be produced in the gastrointestinal tract of the subject. In other aspects of the invention, the Fiaf polypeptide may be produced in part of the subject's body other than the gastrointestinal tract. In still other aspects, the polypeptide is produced ex vivo.

The amount of Fiaf may be increased in a subject by administering an agent comprising a suitable Fiaf polypeptide. Typically, a suitable Fiaf polypeptide is one that can provide radioprotection to a cell of the gastrointestinal system, particularly a villus mesenchymal endothelial cell, when administered to the subject. A number of Fiaf polypeptides known in the art are suitable for use in the present invention. By way of non-limiting example, suitable Fiaf polypeptides and nucleotides are delineated in Table Z. In one embodiment, the Fiaf polypeptide is from the same species as the subject. In another embodiment, the Fiaf polypeptide is from a different species from the subject. In yet another embodiment, both the Fiaf polypeptide and the subject are both mammalian. In still yet another embodiment, both the Fiaf polypeptide and the subject are non-human mammalian. In a further embodiment, both the Fiaf polypeptide and the subject are human.

TABLE Z

| | PubMed | Seq | PubMed | Seq |
|---|---|---|---|---|
| Species | nucleotide ref. | ID NO: | protein ref. | ID NO: |
| Homo | NM_139314 | 1 | NP_001034756 | 15 |
| sapiens | | | | |
| | NM_016109 | 2 | NP_647475 | 16 |
| | NM_001039667.1 | 3 | AAH23647 | 17 |
| Mus | NM_020581 | 4 | AAF86342.1 | 18 |
| Musculus | | | | |
| | AF278699.1 | 5 | AAH21343.1 | 19 |
| Rattus | NM_199115 | 6 | AAH78944.1 | 34 |
| norvegicus | | | | |
| | BC078944.1 | 7 | | |
| Sus scrofa | AY307772 | 8 | | |
| | NM_001038644.1 | 9 | | |
| Bos Taurus | AY192008 | 10 | AAI05517.1 | 20 |
| | BC105516.1 | 11 | | |
| Pan | AY411895 | 12 | | |
| troglodytes | | | | |
| Macaca | XR_012476.1 | 13 | | |
| Mulatta | | | | |
| Canis | XM_533928.2 | 14 | | |
| familiaris | | | | |
| Danio rerio | | | XP_692048.1 | 21 |

Because the sequence and function of Fiaf polypeptides are conserved through evolution, in certain aspects, a polypeptide that is a homolog, ortholog, mimic or degenerative variant of a Fiaf polypeptide is also suitable for use in the present invention. A functional fragment of a Fiaf polypeptide is also suitable for use in the present invention. In particular, the fragment or polypeptide will typically provide radioprotection to a cell of the gastrointestinal system, particularly an endothelial cell, when administered to the subject.

A number of methods may be employed to determine whether a particular fragment, homolog, mimic or degenerative variant possesses substantially similar biological activity relative to a Fiaf polypeptide. Specific activity or function may be determined by convenient in vitro, cell-based, or in vivo assays, such as measurement of radioprotective activity in small intestine tissue. Such assays are commonly known in the art. Generally speaking, such an assay would measure cellular damage in response to varying doses of radiation in the presence of different concentrations of a Fiaf polypeptide. In some embodiments, apoptosis may be used as a measure of cellular damage. A protocol for identifying apoptotic cells in different tissues is described in the examples.

In addition to having a substantially similar biological function, a homolog ortholog, mimic or degenerative variant suitable for use in the invention will also typically share substantial sequence similarity to a Fiaf polypeptide. In addition, suitable homologs, ortholog, mimic or degenerative variants preferably share at least 30% sequence homology with a Fiaf polypeptide, more preferably, 50%, and even more preferably, are greater than about 75% homologous in sequence to a Fiaf polypeptide. Alternatively, peptide mimics of Fiaf could be used that retain critical molecular recognition elements, although peptide bonds, side chain structures, chiral centers and other features of the parental active protein sequence may be replaced by chemical entities that are not native to Fiaf protein yet, nevertheless, confer activity.

In determining whether a polypeptide is substantially homologous to a Fiaf polypeptide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul [(Proc. Natl. Acad. Sci. USA 87, 2264 (1993)]. Such an algorithm is incorporated into the NBLAST and XBLAST programs of Altschul, et al. (J. Mol. Biol. 215, 403 (1990)). BLAST nucleotide searches may be performed with the NBLAST program to obtain nucleotide sequences homologous to a nucleic acid molecule of the invention, Equally, BLAST protein searches may be performed with the XBLAST program to obtain amino acid sequences that are homologous to a polypeptide of the invention. To obtain gapped alignments for comparison purposes, Gapped BLAST is utilized as described in Altschul, et al., (Nucleic Acids Res. 25, 3389 (1997)). When utilizing BLAST and Gapped BLAST programs, the default parameters of the respective programs (e.g., XBLAST and NBLAST) are employed. See http://www.ncbi.nlm.nih.gov for more details.

Fiaf polypeptides suitable for use in the invention are typically isolated or pure and are generally administered as a composition in conjunction with a suitable pharmaceutical carrier, as detailed below. A pure polypeptide constitutes at least about 90%, preferably, 95% and even more preferably, at least about 99% by weight of the total polypeptide in a given sample.

The Fiaf polypeptide may be synthesized, produced by recombinant technology, or purified from cells using any of the molecular and biochemical methods known in the art that are available for biochemical synthesis, molecular expression and purification of the Fiaf polypeptides [see e.g., Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York)].

Expression vectors that may be effective for the expression of Fiaf polypeptides include, but are not limited to, those that function in bacteria (pET and pGEX) and those that function in eukaryotic cells [PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSHIPERV (Stratagene, La Jolla Calif.), and PTET-OFF, PTET-ON, PTRE2, PTRE2-LUC, PTK-HYG (Clontech, Palo Alto Calif.)]. Fiaf polypeptides may be expressed using a constitutively active promoter, e.g., from cytomegalovirus (CMV), Rous sarcoma virus (RSV), SV40 virus, thymidine kinase (TK), or P β-actin genes. Alternatively, they may be expressed using an inducible promoter, e.g., the tetracycline-regulated promoter (Gossen, et al., Proc. Natl. Acad. Sci. USA, 89, 5547 (1992); M. Gossen, et al., Science, 268, 1766 (1995); F. M., Rossi, et al., Curr. Opin. Biotechnol. 9, 451 (1998), commercially available in the T-REX plasmid (Invitrogen); the ecdysone-inducible promoter (available in the plasmids PVGRXR and PIND; Invitrogen); the FK506/rapamycin inducible promoter; or the RU486/mifepristone inducible promoter (F. M. Rossi, et al., supra). In another alternative, they may be expressed using a tissue-specific promoter or the native promoter of the endogenous gene encoding Fiaf from a normal individual.

Commercially available liposome transformation kits (e.g., the PERFECT LIPID TRANSFECTION KIT, available from Invitrogen) allow one with ordinary skill in the art to deliver Fiaf polynucleotides to target cells in culture, and require minimal effort to optimize experimental parameters. Alternatively, transformation may be performed using the calcium phosphate method (F. L. Graham, et al., Virology, 52, 456 (1973), or by electroporation (E. Neumann, et al., EMBO J., 1, 841 (1982)).

A Fiaf peptide can be synthesized using traditional solid-phase methods.

Fiaf polypeptides useful in the practice of the present invention may be formulated into pharmaceutical compositions and administered by any means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In a preferred embodiment, the Fiaf polypeptide is injected into the subject. Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the Fiaf polypeptide may be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms of the Fiaf polypeptide for oral administration may include capsules, tablets, pills, powders, and granules. In such solid dosage forms, the Fiaf polypeptide is ordinarily combined with one or more adjuvants appropriate to the indicated route of administration. If administered via pill, the Fiaf polypeptide may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration, Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions may be prepared from sterile powders or granules having one or more of the carriers or diluents mentioned for use in the formulations for oral administration. Other adjuvants and modes of administration are well and widely known in the pharmaceutical art.

Liquid dosage forms for oral administration may include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that may be combined with the carrier materials to produce a single dosage of the Fiaf polypeptide will vary depending upon the subject and the particular mode of administration. The regimen for treating a subject suffering from gastrointestinal radiosensitivity or at risk for developing gastrointestinal radiosensitivity with a Fiaf polypeptide is selected in accordance with a variety of factors, including the age, weight, sex, diet, and medical condition of the patient, the nature of the radiation therapy (i.e., dose, frequency of administration, treatment area), the route of administration, pharmacological considerations such as the activity, efficacy pharmacokinetic, and toxicology profiles of the particular compounds employed, and whether a drug delivery system is utilized.

In certain embodiments of the invention, the amount of Fiaf may be increased by administering to a subject an agent comprising a suitable nucleotide sequence encoding a Fiaf polypeptide. A suitable nucleotide sequence is one encoding a polypeptide having Fiaf activity, when administered to the subject. A number of nucleotide sequences encoding a polypeptide having Fiaf activity are known in the art and are suitable for use in the present invention. By way of non-limiting example, suitable Fiaf nucleotide sequences are delineated in Table Z, above. Typically, a suitable nucleotide sequence is one encoding a polypeptide that can provide radioprotection to a cell of the gastrointestinal system, particularly a villus mesenchymal endothelial cell, when administered to the subject. Thus, in certain aspects, a nucleotide sequence encoding a polypeptide that is a fragment, homolog, ortholog, mimic or degenerative variant of a Fiaf polypeptide is also suitable for use in the present invention. The subject polypeptide will typically provide radioprotection to a cell of the gastrointestinal system, particularly a villus mesenchymal endothelial cell, when administered to the subject.

In certain embodiments of the invention, an agent can be delivered that specifically activates Fiaf expression: this agent could represent a natural or synthetic compound that directly activates Fiaf gene transcription, or indirectly activates expression through interactions with components of host regulatory networks that control Fiaf transcription. For example, such an agent could be identified by screening natural product and/or chemical libraries using the gnotobiotic zebrafish model described in Rawls, et al., PNAS 101, 4596-4601 (2004) as a bioassay.

In another alternative of this embodiment, Fiaf expression and/or activity may be increased by administering an agent comprising a Fiaf agonist to the subject. In one preferred embodiment, the Fiaf agonist is a peroxisome proliferator-activated receptor (PPARs) agonist. Suitable PPARs include PPARα, PPARβ/δ, and PPARγ. Fenofibrate is another suitable example of a Fiaf agonist. Additional suitable Fiaf agonists and methods of administration are further described in Mandard, et al., J. Biol Chem, 279, 34411 (2004), and U.S. Patent Publication No. 2003/0220373, which are both hereby incorporated by reference in their entirety.

### III. Alteration of the Gastrointestinal Microbiota or Archaeon Population

Another aspect of the present invention provides a method to treat gastrointestinal radiation sensitivity in a subject comprising altering the microbial population or the archaeon population in the subject's gastrointestinal tract. Alternatively, both the microbial population and archaeon population may be altered.

In certain embodiments, the microbiota is altered such that the amount or activity of Fiaf polypeptide is increased, whereby increasing the amount of or activity of Fiaf polypeptide causes a decrease in gastrointestinal radiation sensitivity. In certain embodiments, the presence of microbes that suppress Fiaf expression may be decreased. In certain alternative embodiments, the presence of at least one genera of microbes is decreased. In other alternatives, the presence of at least one species of microbes is decreased. By way of non-limiting example, the microbe may be selected from the genera consisting of Bacteroides.

To decrease the presence of any of the microbes detailed above, methods generally known in the art may be utilized. In one embodiment, a suitable probiotic is administered to the subject. Generally speaking, suitable probiotics include those that alter the representation or biological properties of microbiota populations that are involved in a subject's response to radiation, namely gastrointestinal radiation sensitivity. By way of non-limiting example, suitable probiotics include Lactobacillus and Bifidobacteria, each of which is commercially available from several sources. In another embodiment, microbes that induce Fiaf expression in the subject's gastrointestinal tract may be administered to the subject. In yet another embodiment, selective reduction in the representation of components of the microbiota, i.e., one or more genera or species of bacteria, is achieved by administering an antibiotic to the subject.

In another embodiment, the subject's gastrointestinal archaeon population is altered such that the amount or activity of Fiaf polypeptide is increased, whereby increasing the amount of or activity of Fiaf polypeptide causes a decrease in gastrointestinal radiation sensitivity. Typically, the presence of at least one genera of archaeon that resides in the gastrointestinal tract of the subject is decreased. In most embodiments, the archaeon is generally a mesophilic methanogenic archaea. In one alternative of this embodiment, the presence of at least one species from the genera Methanobrevibacter, is decreased. In another alternative embodiment, the presence of Methanobrevibacter smithii is decreased. In yet another embodiment, the presence of a combination of archaeon genera or species is decreased. By way of non-limiting example, the presence of Methanobrevibacter smithii and Methanosphaera stadtmaniae is decreased.

To decrease the presence of any of the archaeon detailed above, methods generally known in the art may be utilized. In one embodiment, a compound having anti-microbial activities against the archaeon is administered to the subject. Non limiting examples of suitable anti-microbial compounds include metronidzaole. In yet another embodiment, a hydroxymethylglutaryl-SCoA reductase inhibitor is administered to the subject. Non limiting examples of suitable hydroxymethylglutaryl-SCoA reductase inhibitors include lovastatin. Alternatively, the diet of the subject may be formulated by changing the composition of glycans (e.g., fructose-containing oligosaccharides) in the diet that are preferred by polysaccharide degrading bacterial components of the microbiota (e.g., Bacteroides spp) when in the presence of mesophilic methanogenic archaeal species such as Methanobrevibacter smithii.

### IV. Combination Therapy

Another aspect of the invention encompasses a combination therapy to treat gastrointestinal radiation sensitivity in a subject. In one embodiment, the invention encompasses a composition for decreasing gastrointestinal radiation sensitivity. As discussed above, gastrointestinal radiation sensitivity manifests itself as the condition known as radiation enteritis, which encompasses various disorders, including diarrhea, dehydration, malabsorption, abdominal pain, rectal pain, weight loss, nausea, vomiting, fatty stools, bloody stools, and frequent urges to have a bowel movement.

Typically, the composition comprises a first agent that increases the amount or activity of a Fiaf polypeptide and a second agent that ameliorates a symptom of radiation enteritis, specifically one or more of the gastrointestinal disorders involved in enteritis. Suitable agents that increase the amount or activity of Fiaf polypeptides are detailed in section (II). Radiation enteritis and the disorders associated with it can be treated with a variety of agents, including an antidiarrheal agent, a rehydration therapy, a nutritional supplement, an analgesic, an antinausea agent, and an antispasmodic agent. More specifically, treatments for these disorders include bismuth subsalicylate, diphenoxylate, atropine sulphate, paregoric, cholestyramine, phenobarbital, loperamide hydrochloride, prochlorperazine, promethazine hydrochloride, metoclopramide hydrochloride, trimethobenzamide hydrochloride, ondansetron hydrochloride, aspirin, naproxen, a solution comprising glucose and electrolytes, ibuprofen, acetaminophen, and a multi-vitamin and multi-mineral supplement, Chronic radiation enteritis may be treated with the above-described agents as well as surgery in some, more severe cases.

In still another embodiment, a second agent that modulates the intestine's mesenchymal apoptotic response to γ-radiation may be included in the combination therapy. In one alternative of the embodiment, fibroblast growth factor is administered. Administration of fibroblast growth factor-2 typically attenuates intestinal mesenchymal apoptosis induced by total body irradiation (TBI) and reduces mortality due to TBI. In another embodiment, vascular endothelial growth factor is administered. Pre-treatment with vascular endothelial growth factor typically provides partial protection against lethal radiation enteritis. In yet another alternative of this embodiment, an inhibitor of acid sphingomyelinase is administered. Loss of acid sphingomyelinase activity has been shown to reduce TBI-induced mesenchymal apoptosis in the intestine and reduce mortality.

In an additional embodiment, the composition of the invention may be administered with a chemotherapeutic agent. As will be appreciated by the skilled artisan, the type of chemotherapeutic agent can and will varying depending upon the type of cancer being treated and its stage of progression. Non-limiting examples of chemotherapeutic agents include alkylating agents, such as cisplatin, carboplatin, or oxaliplatin, antimetabolites, i.e. agents that prevent nucleic acid bases from incorporating into DNA during the “S” phase of the cell cycle, anthracyclines, plant alkaloids, i.e. agents that block microtubule function such as vinca alkaloids and taxanes, topoisomerase inhibitors such as irinotecan, topotecan, amsacrine, etoposide, or teniposide, and other antitumor agents

Selection of the appropriate agents for use in combination therapy may be made by one of ordinary skill in the art, according to conventional pharmaceutical principles. The combination of therapeutic agents may act additively to decrease gastrointestinal radiation sensitivity. Using this approach, one may be able to achieve therapeutic efficacy with lower dosages of each agent, thus reducing the potential for adverse side effects.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means. Typically, the compositions comprise an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.).

As will be appreciated by the skilled artisan, the actual effective amounts of compound described herein can and will vary according to the specific composition being utilized, the mode of administration and the age, weight and condition of the subject. Dosages for a particular individual subject can be determined by one of ordinary skill in the art using conventional considerations. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

Moreover, the timing of the administration of the first agent (i.e., the agent that increases the amount or activity of Fiaf) in relation to the administration of the second agent (i.e., the agent that ameliorates at least one symptom in gastrointestinal radiosensitivity) may also vary from subject to subject. In one embodiment, the first and second agents may be administered substantially simultaneously, meaning that both agents may be administered to the subject at approximately the same time. For example, the first agent is administered during a continuous period beginning on the same day as the beginning of the administration of the second agent and extending to a period after the end of the second agent. Alternatively, the first agent and second agent may be administered sequentially, meaning that they are administered at separate times during separate treatments. In one embodiment, for example, the first agent is administered during a continuous period beginning prior to administration of the second agent and ending before administration of the second agent. Of course, it is also possible that the first agent may be administered either more or less frequently than the second agent. Moreover, it will be apparent to those skilled in the art that it is possible, and perhaps desirable, to combine various times and methods of administration in the practice of the present invention. For example, the first and second agent, in some instances, may be advantageously administered to the subject prior to radiation treatment. Alternatively, in other instances, the first and second agent may be administered to the subject after or at substantially the same time as radiation treatment.

### DEFINITIONS

The term “agonist” refers to a molecule that enhances or increases the biological activity of a Fiaf polypeptide. Agonists may include proteins, peptides, nucleic acids, carbohydrates, small molecules (e.g., such as metabolites), or other compounds or compositions that modulate the activity of a Fiaf polypeptide either by directly interacting with the polypeptide, the nucleic acid sequence of the polypeptide, or by acting on components of the biological pathway in which Fiaf participates.

The term “altering” as used in the phrase “altering the microbiota population” is to be construed in its broadest interpretation to mean a change in the representation of microbes in the gastrointestinal tract of a subject. The change may be a decrease or an increase in the presence of a particular microbial species.

BMT stands for bone marrow transplantation.

CONV-D stands for conventionalization of germ free animals with a gut microbiota harvested from conventionally-raised donor animals.

CONV-R stands for conventionally raised, i.e., acquiring microbes beginning at birth.

“Conservative amino acid substitutions” are those substitutions that are predicted to least interfere with the properties of the original protein, i.e., the structure and especially the function of the protein is conserved and not significantly changed by such substitutions.

An “effective amount,” “therapeutically effective dose,” or a “therapeutically effective amount” is an amount (or dose) that is intended to quantify the amount of agent that will achieve the goal of a decrease in gastrointestinal radiosensitivity.

Fiaf stands for fasting-induced adipose factor, also lanolin as angiopoietin-like protein 4 or angpt14.

A “gene” is a hereditary unit that has one or more specific effects upon the phenotype of the organism and that can mutate to various allelic forms.

GF stands for germ free.

Gray (Gy) is the international unit for measuring the absorbed dose of radiation.

iNOS stands for inducible nitric oxide synthase.

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

A “nucleic acid” is a nucleotide polymer of DNA or RNA, it consists of purine or pyrimidine base, e.g. with associated pentose sugars, and phosphate groups.

PPAR stands for peroxisome proliferator-activator receptor.

“Peptide” is defined as a compound formed of two or more amino acids, with an amino acid defined according to standard definitions.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzy1ethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

A “polypeptide” is a polymer made up of amino acids.

“Protein” is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

TBI stands for total body irradiation.

TEM stands for transmission electron microscopy.

The term “treat” or “treatment” as used herein, includes providing therapy according to the methods of the invention to a subject known to have gastrointestinal radiosensitivity, preventing the onset of a clinically evident case of gastrointestinal radiosensitivity, or preventing the onset of a preclinically evident stage of gastrointestinal radiosensitivity in a subject. Consequently, this definition includes prophylactic treatment.

A “vector” is a self-replicating DNA molecule that transfers a DNA segment to a host cell.

WT stands for wild-type.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate the invention. In the examples, the following materials and methods were used.

### Materials and Methods

Mice—CONV-R wild-type (wt) FVB/N mice were purchased from Taconic. C57BL/6J (B6) wt, Rag1−/−, iNOS−/−, and FVB/N Tie2-GFP (green fluorescent protein) transgenic mice were obtained from Jackson Laboratories. Male and female FVB/N and B6 wt mice were re-derived as GF and conventionalized, or were colonized with one or two components of the normal gut microbiota (25). GF and CONV-D Fiaf−/− mice (hybrid C57Bl/6J: 129/SvJ background) were produced according to ref. 3. Sca1-EGFP knock-in mice (originally on a B6:129 hybrid background but backcrossed to B6 to generation N7) were generously supplied by Timothy Graubert (Washington University, St. Louis, Mo.).

GF, CONV-D, and specified pathogen-free CONV-R wt and genetically engineered mice were maintained under a 12 h light cycle, and fed an autoclaved standard rodent chow diet (B&K; Zeigler Brothers, Gardners, Pa.) plus sterilized tap water ad libitum. GF and CONV-D mice were housed in plastic gnotobiotic isolators (25). All experiments involving mice were performed using protocols approved by the Washington University Animal Studies Committee.

TBI and bone marrow transplantation (BMT) of GF mice—An irradiator pie-plate for housing animals was sterilized (aerosolized chlorine dioxide; Clidox-S; Pharmacal Research Labs, Waterbury, Conn.) and passed into a flexible film gnotobiotic isolator where it was fitted with a sterile fiberglass filter top (0.6 cm thick Afs-4 filter media, Class Biologically Clean, Madison, Wis.) to allow air, but not microbes, to pass through. GF mice in this container were removed from the isolator, and treated in a Mark I ¹³⁷Cs irradiator (Shepherd; 106 cGy/min for a total dose of 10 to 22 Gy). After irradiation, the container was placed in the gnotobiotic isolator's entry port. Following sterilization (aerosolized Clidox-S; 20 min), the inner door of the port was removed, the container pulled through, and the port was closed. Mice were then returned to their cages within the isolator.

For transplantation of unfractionated bone marrow, 8-12 week-old strain-, gender, and genotype-matched donor mice were killed, sprayed with 70% ethanol, and their femors and tibias recovered by dissection. Using a laminar flow hood, the contents of these long bones were flushed into Hanks' Balanced Salt Solution containing 100 U/mL penicillin and 100 μg/mL streptomycin. Fibrous debris was allowed to settle by gravity, and the cell-enriched supernatant was aspirated and centrifuged (2000×g for 10 min at 25° C.). The resulting cell pellet was resuspended in 1-2 mL PBS supplemented with 100 U/ml penicillin and 100 μg/mL streptomycin. Cells were counted (hemocytometer) and aliquoted into sterile screw-cap tubes. One donor mouse provided cells for 3-4 recipients: each recipient received ˜5×10⁶ antibiotic-sterilized cells.

For BMT of GF mice, 1 mL syringes and 30 gauge needles were autoclaved in a transporting cylinder and shuttled into the gnotobiotic isolator through its port. A sterile screw cap tube, containing Rodent Anesthesia Cocktail [Ketamine (100 mg/mL), Zylazine, (20 mg/mL), Acepromazine (10 mg/mL)] that had been passed through a 0.2 μm filter, was sterilized in the port of the gnotobiotic isolator and then passed into the isolator. GF mice received an intraperitoneal dose of 0.25 mL of the anesthesia cocktail at least 6 h, but not more than 18 h after TBI. A screw-cap tube containing the donor bone marrow cells was passed into the isolator, using the same procedure employed for the anesthesia cocktail. Once in the isolator, the tube was rinsed in sterile water before cells were collected in a 1 mL syringe and a 100-200 μL aliquot delivered to the recipient via retro-orbital injection.

To confirm preservation of the GF state after TBI-BMT, fecal samples from mice in the isolators were cultured in Brain-Heart Infusion broth, Sabouraud dextrose broth, and Nutrient broth (VWR Scientific) at 37° C. and 42° C. under aerobic and anaerobic conditions. Spleens were dissected from mice using sterile techniques, homogenized in 1 mL PBS, and serial dilutions (in PBS) were plated on Brain-Heart Infusion blood agar. The plates were subsequently cultured under aerobic and anaerobic conditions.

Statistics—Log-rank analysis was used to compare survival curve data. The significance of observed differences in intestinal mesenchymal apoptosis among members of various treatment groups was assessed by using two-tailed Student's t-test or one-way or two-way ANOVA, followed by Bonferroni post-hoc comparison.

Histochemistry and immunohistochemistry—The small intestines of irradiated and non-irradiated mice were each divided into 16 equal-length segments (proximal-most segment designated as 1). Segments 11-14 were flushed with PBS, cut open along their cephalocaudal axes, pinned on wax-covered plates, and incubated overnight in 10% neutral buffered formalin at 4° C. Fixed specimens were washed in 70% ethanol, mounted in 2% agar, embedded in paraffin, and 7 μm-thick sections were cut for subsequent staining with hematoxylin and eosin.

For immunohistochemistry, the dissected intestinal segments were fixed overnight at 4° C. in freshly-prepared 4% paraformaldehyde (in PBS), washed in PBS, incubated in 10% sucrose/90% PBS (3 h at 4° C.), immersed in 20% sucrose/10% glycerol/70% PBS (overnight at 4° C.), and then frozen in Optimal Cutting Temperature (OCT) TissueTek compound (VWR Scientific) prior to cryosectioning (44). Rat monoclonal antibodies to CD31 and CD45 (BD Pharmingen) were diluted 1:100 in blocking buffer (PBS/1% bovine serum albumin/0.1% Triton X-100) prior to use. Rabbit anti-GFP was obtained from Molecular Probes, and used at a dilution of 1:1000. Secondary Alexa Fluor488 or -594 conjugated antibodies to rat or rabbit Igs (Molecular Probes; 1:100) were used to visualize antigen-antibody complexes. Nuclei were stained with 4′,6′-diamidino-2-phenylindole (DAPI; 40 ng/mL blocking buffer).

For terminal deoxynucleotidyl transferase-mediated dUTP nick-end labeling (TUNEL) assays, the small intestine was dissected as described above, immediately frozen in OCT, cryosectioned, and treated with reagents supplied in the In situ Cell Death Detection Kit (Roche Diagnostics). The number of mesenchymal cells that were both TUNEL-positive and DAPI-positive were scored in each well-oriented villus section (≧100 sectioned villi surveyed/animal).

To visualize the capillary network underlying the small intestinal epithelium, fluorescein isothiocyanate (FITC)-labeled Bandeiraea simplicifolia-1 lectin (BS-1; Sigma-Aldrich) was injected retro-orbitally (25 mg/kg body weight) 30 min before animals were killed. For 3-D reconstructions of the capillary network, 60 μm-thick sections were scanned with a Zeiss ApoTome microscope at an optical section thickness of 1 μm, and the collected serial images rotated at 10° intervals.

Transmission electron microscopy (TEM). Distal small intestinal segments from 8-10 week old B6 male mice, or B6:129/SvJ Fiaf+/+ and Fiaf−/− littermates, were rapidly dissected after the animals were killed. The segments were flushed with PBS, cut open along their cephalocaudal axes, pinned on wax, and fixed overnight at 4° C. in a solution containing 2% paraformaldehyde, 2.5% glutaraldehyde, and 1% tannic acid (prepared in 0.1 M cacodylic acid, pH 7.2). Following multiple rinses in 0.1 M cacodylic acid, samples were fixed in 1% osmium tetroxide/0.1 M cacodylic acid for 1 h, and then stained for 1 h en bloc in 1% uranyl acetate (prepared in distilled, deionized water). Samples were subsequently dehydrated through a series of graded ethanols and propylene oxide, then infiltrated with and embedded in monomeric Embed 812 (Electron Microscopy Sciences, Hatfield, Pa.). 75 nm-thick sections were cut from each block, placed on grids, stained with Reynolds' lead citrate/1% uranyl acetate, and viewed under a Hitachi H7500 transmission electron microscope.

RNA isolation and real-time qRT-PCR—Small intestines were harvested from 8 week old mice belonging to one of four groups: GF or CONV-R, untreated or killed 4 h after 16 Gy TBI (n=4 mice/group). Segment 14 from each animal's small intestine was snap frozen in liquid nitrogen and RNA was isolated (QIAGEN RNeasy kit with DNase I). RNA was reverse-transcribed using SuperScript II (Invitrogen) and oligo dT₁₈(New England Biolabs). qRT-PCR assays were performed using the primers listed in Table S1, SYBR green detection, and a Stratagene Mx3000P thermocycler. Data were analyzed using the ΔΔC_{T} method (45).

TABLE S1

Primer pairs used for real-time qRT-PCR.

| | | SEQ | | SEQ |
|---|---|---|---|---|
| Tran- | Forward | ID | Reverse | ID |
| script | primer | NO: | Primer | NO: |
| INOS | 5′- | 22 | 5′-CATTGGAAGTGAAGCGTTTCG | 23 |
| | CAGCTGGGCTGTACAAACC | | | |
| | TT | | | |
| Vegf-A | 5′- | 24 | 5′-ATCCGCATGATCTGCATGG | 25 |
| | AGTCCCATGAAGTGATCAA | | | |
| | GTTCA | | | |
| Fgf-2 | 5′- | 26 | 5′-CCCGTTTTGGATCCGAGTTT | 27 |
| | GTCACGGAAATACTCCAGT | | | |
| | TGGT | | | |
| ASMase | 5′- | 28 | 5′-TGTGAAGGGCATCAGCTGGTAA | 29 |
| | AGAGCACTCCTGTCAATGG | | | |
| | CTT | | | |
| Fiaf | 5′- | 30 | 5′-TGGCCGTGGGCTCAGT | 31 |
| | CAATGCCAAATTGCTCCAAT | | | |
| | T | | | |
| L32 | 5′- | 32 | 5′-TCTGGGTTTCCGCCAGTTT | 33 |
| | CCTCTGGTGAAGCCCAAGA | | | |
| | TC | | | |

Administration of neutralizing antibodies—Polyclonal goat anti-mouse Vegf-A neutralizing antibody (antigen: recombinant mouse VEGF₁₆₄) was purchased from R&D Systems. Forty micrograms were given retro-orbitally 12 h prior to, 20 μg 1 h prior to, and 20 μg 1 h after 16 Gy TBI. Mouse monoclonal antibody to bovine Fgf-2 (known to cross react with the mouse ortholog) was purchased from Upstate Cell Signaling Solutions. A single retro-orbital injection of 100 μg was given 3 h prior to TBI. These previously characterized neutralizing antibodies were administered to mice at doses that were equal to or greater than those previously shown to be effective in in vivo angiogenesis assays (46-49).

### Example-1

The results depicted in example 1 demonstrate that the small intestine of GF mice is radioresistant. In most mouse strains, TBI ultimately causes death in CONV-R animals by bone marrow failure at doses of 10 Gy, and death from a combination of bone marrow failure and radiation enteritis at doses ≧15 Gy. A protocol was developed for exposing 8-12 week old male and female GF FVB/N mice to TBI in a ¹³⁷Cs irradiator without compromising their GF status as detailed above. Age- and gender-matched GF and CONV-R mice were fed the same autoclaved chow diet and treated with identical levels of irradiation. To rescue the animals from death due to bone marrow failure, bone marrow transplantation (BMT) was performed by retro-orbital injection of antibiotic-sterilized unfractionated marrow harvested from age-, gender- and strain-matched CONV-R donors. To confirm the efficacy of retro-orbital delivery of bone marrow, control animals were transplanted after 10 Gy TBI: >95% of GF and CONV-R mice (n=23) survived ≧60 d and all GF mice remained sterile as judged by periodic culture of their stool (and cecal contents at the time of sacrifice).

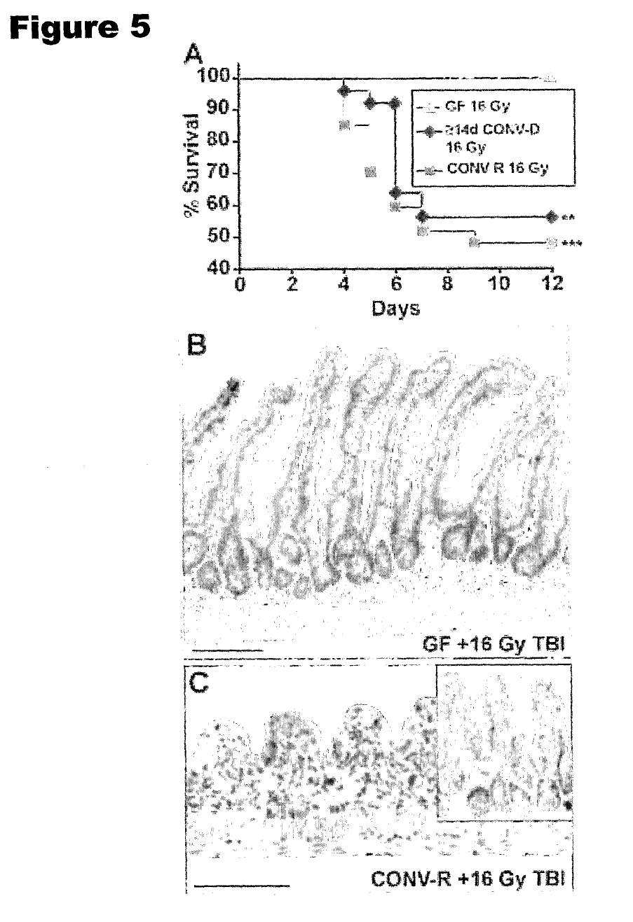

All GF mice treated with a higher TBI dose (16 Gy) followed by BMT survived for ≧40 d (n=18). They ultimately succumbed to dental malocclusion due to the deleterious effects of irradiation on growth of the tooth bud. In contrast, 52% of CONV-R mice treated with 16 Gy followed by BMT died within 7 d (n=27; p<0.001 compared to GF; see FIG. 5A). The role of the microbiota in imparting this increased radiosensitivity was confirmed by colonizing GF mice for 14 d with a cecal microbiota harvested from CONV-R donors. When the resulting conventionalized (CONV-D) mice were treated with 16 Gy TBI then BMT, 44% died within 7 d [n=25, p<0.005 compared to GF; no significant difference compared to CONV-R animals p=0.40)]. A 14 d colonization of GF mice with Bacteroides thetaiotaomicron (sequenced strain VPI-5482) and/or Escherichia coli (sequenced strain MG1655)—representing prominent obligate and facultative anaerobes, respectively, in the normal human and mouse microbiotas—did not affect their radioresistant phenotype after 16 Gy TBI, even though the density of their colonization was similar to that achieved after conventionalization with a complete cecal microbiota from CONV-R donors (10¹¹-10¹² CFU/mL cecal contents; n=8 bi-associated and 4 monoassociated mice). This result indicated that there is microbial specificity to the lethal radiosensitive phenotype imparted by the microbiota, and that one or more other species are required in lieu of, or in addition to these two.

A histological analysis was performed to determine if the different survival times of GF and colonized mice was due to a differential response of their small intestines to 16 Gy TBI. OF FVB/N mice sacrificed up to 14 d after TBI-BMT had no histological evidence of radiation enteritis (see FIG. 5B). In contrast, their CONV-R or CONV-D counterparts had the classical hallmarks of this injury response when they were sacrified immediately after becoming moribund 5-7 d after TBI, whether or not they had received a BMT (n=5-8 mice/group; FIG. 5C). The relative radioresistance of GF compared to CONV-D or CONV-R intestines disappeared at 20 Gy: all GF animals receiving this dose succumbed by 9 d after TBI-BMT with radiation enteritis (n=8; data not shown).

CONV-R and CONV-D mice who received 14 Gy (a lethal TBI dose just below the threshold for developing radiation enteritis) and those that received 16 Gy (above the threshold) had no statistically significant differences in the mean number of aerobic or anaerobic bacteria in their spleens (5×10⁶ colony forming units (CFU)/spleen at the lower dose versus 1.3×10⁷ CFU/spleen at the higher dose; n=5 mice/treatment group; p=0.49 by two-tailed Student's t-test, Note that non-irradiated controls had ≦10 CFU/spleen). Thus, radiation-induced systemic infection is not sufficient to produce the histologic features of radiation enteritis.

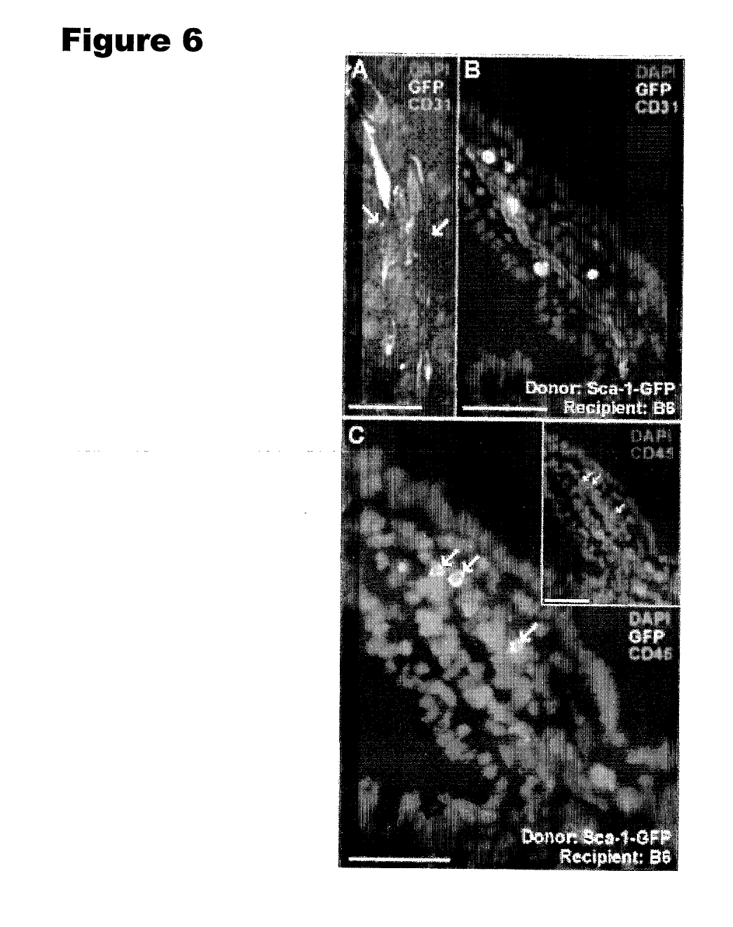

Bone marrow from Tie2-GFP donors (transgene expression marks mucosal endothelial cells; (26)) or Sca-1-EGFP knock-in donors (locus marks all hematopoietic derivatives plus endothelial cells; FIG. 6A), was transplanted into GF, CONV-D, or CONV-R recipients treated with a dose of TBI that does not produce radiation enteritis (10 Gy). Six or twelve weeks later, very few GFP⁺ cells were observed in the villus core mesenchyme of syngeneic recipient mice that received bone marrow from Tie2-GFP donors. Irradiated recipients of marrow from Sca-1-EGFP donors also did not have detectable GFP⁺ endothelial cells in their villus core mesenchyme (FIG. 6B). However, there was extensive population of this mesenchyme with GFP+/CD45+ cells (CD45 is pan-leukocyte marker), as well as GFP+/B220+ B-cells and GFP+/CD3+ T-cells (FIG. 6B).

In view of the forgoing results, it was demonstrated that (i) mice harboring a normal microbiota exhibit enhanced intestinal radiosensitivity compared to GF animals, and this is responsible for their increased mortality after TBI-BMT; (ii) BMT cannot rescue lethality due to radiation enteritis; and (iii) indigenous microbes alter the radioresponsive phenotype of the intestine independent of systemic infection, or functions that are provided by transplanted, bone marrow-derived, villus mesenchymal cells.

### Example-2

The results depicted in Example 2 illustrate that the microbiota increases the sensitivity of mesenchymal endothelial and immune cells to TBI-induced apoptosis.

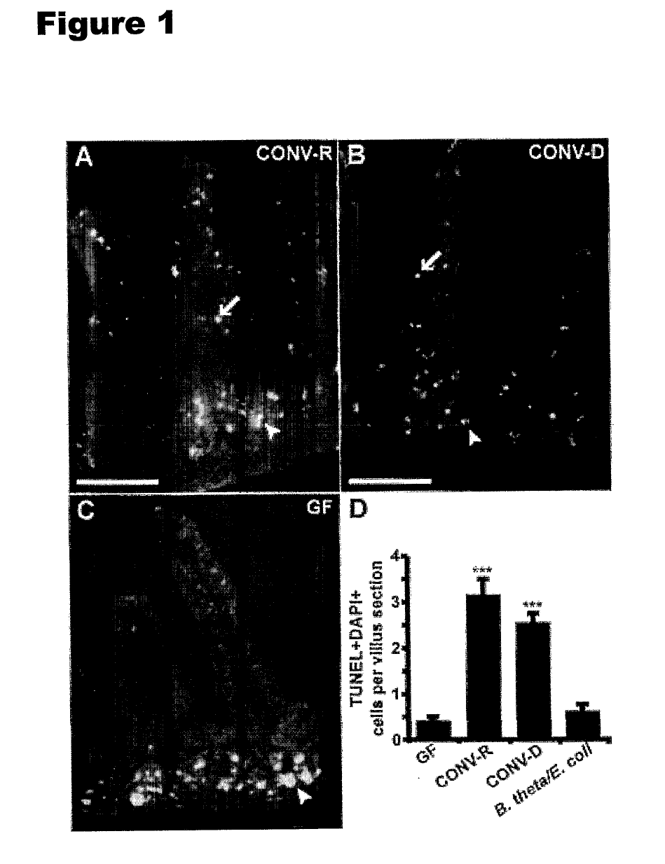

To further define differences in the radiosensitivity of GF and colonized mice, GF, CONV-D and CONV-R FVB/N animals were treated with 16 Gy TBI, killed 4 h later, and apoptotic (TUNEL-positive) cells were scored in cryosections prepared from their distal small intestines (n=4-5 mice/treatment group). The crypts of Lieberkühn, which contain the multipotential small intestinal stem cell and its differentiating descendants (oligo-potential lineage progenitors and a transit amplifying population) exhibited extensive epithelial apoptosis in all three groups of mice, as did cells in the mesenchymal cores of small intestinal villi in CONV-R and CONV-D animals. In contrast, the number of apoptotic cells (i.e. those having TUNEL-positive, DAPI-stained nuclei) in the mesenchymal cores of GF mice was significantly less (FIGS. 1A-D). Consistent with their post-TBI-BMT survival curves, there were no significant quantitative differences in the mesenchymal apoptotic response following 16 Gy TBI in GF versus B. thetaiotaomicron/E. coli bi-associated mice (FIG. 1D).

Time-course studies involving conventionalization of GF mice for 1, 2, 7, and 14 d prior to 16 Gy TBI-BMT revealed that there were no significant differences in the number of TUNEL+−DAPI+ mesenchymal cells/villus section compared to non-irradiated GF controls until 14 d (n=3 mice assayed/treatment group/time point). Administering considerably higher doses of TBI (22 Gy) to GF animals prior to TBI, or varying the interval between 16 Gy TBI and their time of sacrifice from 1 h to 24 h did not yield a statistically significant increase in the number of apoptotic mesenchymal cells (data not shown). The development of radiation enteritis in GF mice in the absence of a significant mesenchymal apoptotic response 4 h after 22 Gy TBI is consistent with the notion, gleaned from previous studies of CONV-R mice, that mesenchyme-independent, epithelial-based mechanisms underlie mucosal injury at these exceptionally high doses (28, 29).

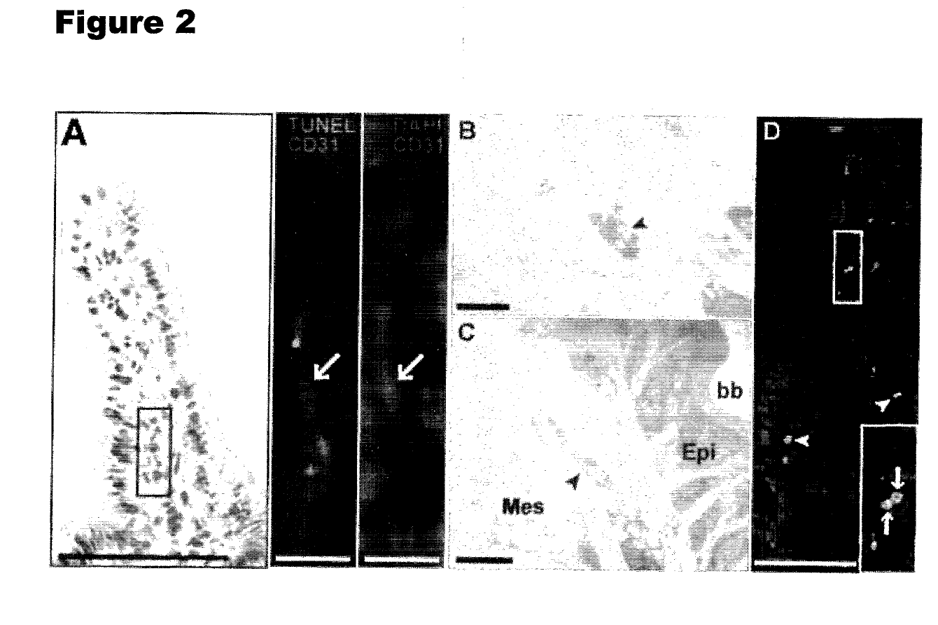

Multi-label immunohistochemical analysis using antibodies specific for CD31 (PECAM-1), plus transmission electron microscopic (TEM) studies established that endothelial cells comprise a subset of the apoptotic villus mesenchymal population that appears in CONV-R mice 4 h after 16 Gy TBI (FIGS. 2A,B). In contrast, endothelial cells in the villus mesenchyme of treatment-matched GF mice had normal ultrastructural morphology (FIG. 2C; n≧30 villus sections surveyed by TEM/animal, corresponding to ≧200 endothelial cells).

Administering 16 Gy TBI to wt CONV-R and CONV-D mice also increased the number of CD45+ apoptotic cells in the mesenchyme (FIG. 2D). Many of these cells were lymphocytes as judged by their reaction with antibodies to B220 or CD3 (data not shown).

Recent studies of CONV-R mice showed that progression to radiation enteritis correlates with radiation-induced endothelial apoptosis (28, 30). The in vivo studies of gnotobiotic mice described in this invention link the microbiota to both radiation enteritis and intestinal endothelial apoptosis.

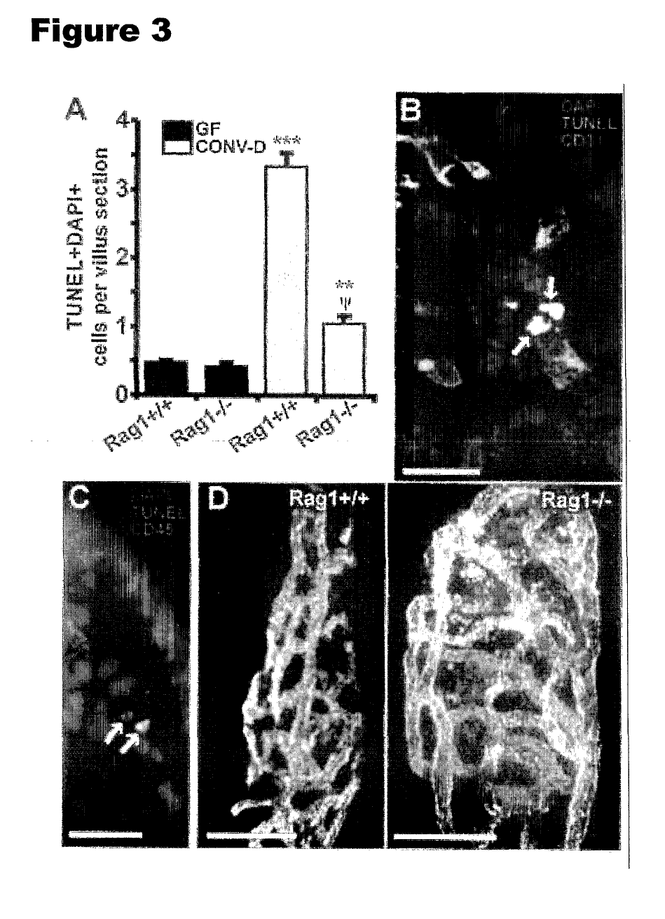

A 14 d conventionalization of GF B6 Rag1−/− mice that lack mature lymphocytes, followed by 16 Gy TBI, produced a statistically significant increase in mesenchymal cell death compared to irradiated GF Rag1−/− controls (FIG. 3A). Nevertheless, these CONV-D B6 Rag1−/− mice had significantly fewer villus mesenchymal apoptotic cells than age- and treatment-matched wt B6 animals (n=4-6 mice/group; FIG. 3A). Apoptotic CD31+ endothelial cells, as well as CD45+ leukocytes, were observed in the villus mesenchymal cores of these CONV-D Rag1−/− mice (FIGS. 3B,C), just as they were in irradiated CONV-D wt littermates. The differences in mesenchymal apoptosis between CONV-D Rag1 knockout and wt animals were not attributable to differences in the complexity of the capillary network that underlies their small intestinal villus mucosa: intravenous injection of FITC-labeled Bandeiraea simplicifolia-1 lectin, which binds endothelial cells, followed by confocal microcopy and 3D reconstruction of serial digital images revealed no appreciable differences in vascular phenotypes between the two groups of animals (FIG. 3D).

Taken together, these results demonstrate that (i) lymphocytes comprise a portion of the villus mesenchymal population of TBI-induced apoptotic cells in wt mice with a microbiota, (ii) a subset of the CD45+ cells that populate the villus mesenchyme of CONV-D Rag1−/− mice is sensitive to TBI-induced apoptosis, and (iii) mature B- and T-cells are not required for induction of endothelial apoptosis.

These studies of Rag1 knockout mice were extended by administering 16 Gy TBI to CONV-D B6 wt and Rag1−/− littermates, followed by BMT with genotype and gender-matched marrow. All mice in each conventionalized group died within 7α following TBI-BMT, and exhibited histologic evidence of radiation enteritis (n=8-10 mice/group; data not shown. This result indicates that the microbiota can confer sensitivity to lethal radiation enteritis in animals that lack mature lymphocytes.

### Example 3

Example 3 demonstrates that Fiaf conveys radioresistance to villus endothelial cells in GF mice.

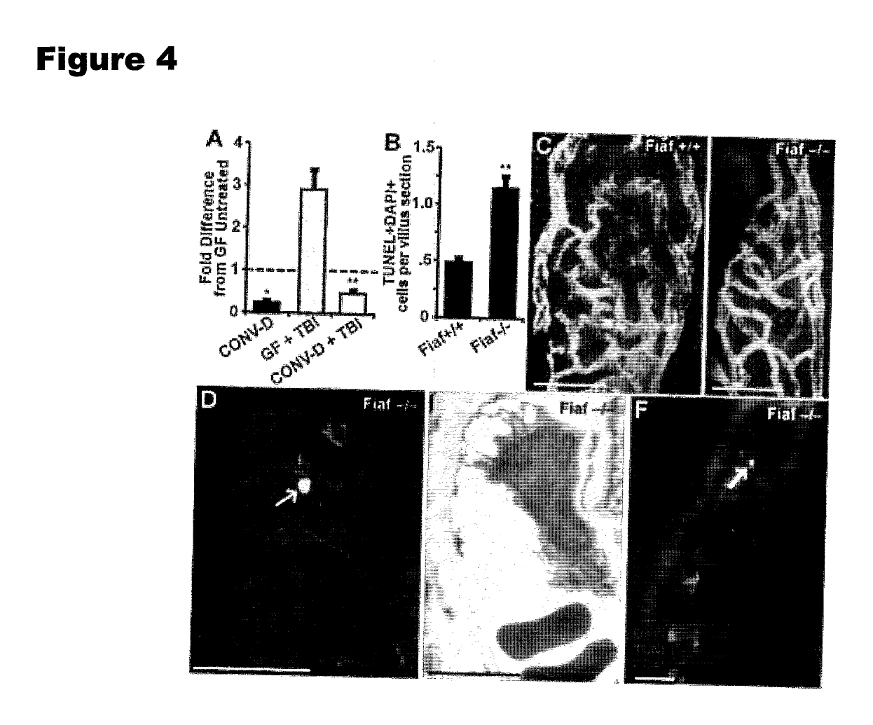

Fiaf is predicted to be composed of coil-coiled and fibrinogen-like domains: it is known to be glycosylated, proteolytically processed, and secreted from several mouse tissues, including the intestinal epithelium (31-34). Fiaf expression is selectively and significantly down-regulated in the gut epithelium by conventionalization of B6 GF mice (4.4±0.5 fold; n=4 mice per group; p<0.05; FIG. 4A and (3)). This is an evolutionarily conserved response: e.g., it occurs during conventionalization of GF zebrafish (35). Fiaf is a physiologically important circulating inhibitor of lipoprotein lipase: studies of GF and CONV-D wt and Fiaf−/− mice indicate that Fiaf is a key mediator of the microbiota's ability to promote host storage of energy, extracted from otherwise indigestible dietary polysaccharides, in adiposes (3). Fiaf also supports endothelial survival (31) and vascular sprouting in vitro (36) through unknown mechanisms. Given its suppression in the gut epithelium by the microbiota, Fiaf was evaluated to determine whether this protein plays a role in imparting radioresistance to villus core mesenchymal endothelial cells in GF animals.

Four hours after 18 Gy of TBI, the number of TUNEL+−DAPI+ mesenchymal cells per villus section was 2.4±0.2 fold greater in 6-10-week old GF Fiaf−/− mice compared to their treatment-matched wt littermates (p<0.01; n=9 mice/group; FIG. 4B). Immunohistochemical and TEM analyses confirmed the presence of CD31+ and CD45+ apoptotic cells in the small intestinal mesenchyme of these irradiated GF Fiaf−/− mice (e.g., FIG. 4D-F). This difference was not attributable to differences in the complexity of their submucosal villus capillary network: network density was similar in untreated GF knockout and wt animals (FIG. 4C).

The reduced expression of Fiaf produced by a 14 d conventionalization of GF wt mice is also evident 4 h after 16 Gy TBI (n=4 mice per group, p<0.01; FIG. 4A). Thus, in the colonized state, Fiaf has a minimized capacity to provide radioprotection to endothelial cells and lymphocytes in wt mice. This would explain why Fiaf affords radioprotection in GF wt mice (there is no microbial suppression of its production), and why there was no significant difference in magnitude of TBI-induced mesenchymal apoptosis between CONV-D wt mice (Fiaf suppressed state) and their CONV-D Fiaf−/− littermates (data not shown).

GF Fiaf−/− mice do not display an increased likelihood for developing lethal radiation enteritis compared to GF wt littermates, as judged by histological studies and the absence of differences in the survival curves of animals given 16-18 Gy TBI followed by gender and genotype-matched BMT (n=10 animals/group; data not shown). This suggests that other factors operate in collaboration with, or independent of Fiaf, to convey these phenotypes. Inducible nitric oxide synthase (iNOS) does not appear to be one of these factors. Nitric oxide (NO) is a known regulator of endothelial survival: at nanomolar concentrations, it is cytoprotective while at micromolar concentrations (achieved with inflammatory stimuli) it is cytotoxic (37, 38). qRT-PCR analysis indicated that 4 h after 16 Gy TBI, iNOS mRNA levels are higher (8.5±1.9-fold) in the distal small intestines of 8 week-old B6 wt CONV-D mice compared to their GF counterparts (n=4 mice/group; p<0.001). Fiaf deficiency does not affect the magnitude of these TBI-induced differences in iNOS expression (data not shown). Moreover, analysis of CONV-R iNOS−/− mice revealed that loss of this NO-producing enzyme had no detectable effect on the extent of villus mesenchymal apoptosis produced by 16 Gy of TBI (n=7 mice/group).

Pharmacologic and genetic studies have revealed that three other factors modulate the intestine's mesenchymal apoptotic response to γ-irradiation. Administration of fibroblast growth factor-2 (Fgf-2) decreases TBI-associated mortality and attenuates TBI-induced intestinal mesenchymal apoptosis in CONV-R wt mice (28, 39). Pre-treatment with vascular endothelial growth factor (Vegf-A) provides partial protection against lethal radiation enteritis (39). CONV-R mice homozygous for a null allele of the gene encoding ASMase (acid sphingomyelinase) have reduced TBI-induced gut mesenchymal apoptosis and lethality compared to their CONV-R wt littermates (28). However, qRT-PCR studies of GF and CONV-D wt and Fiaf knockout mice prior to and 4 h after 16 Gy TBI revealed that neither conventionalization, TBI, nor Fiaf deficiency produces a significant effect on the levels of small intestinal expression of Fgf2, Vegf-A, or ASMase mRNAs (n=4 mice/group; data not shown). Moreover, when a well-characterized neutralizing antibody was administered to mouse Vegf-A, at doses known to block the effects of this protein in in vivo angiogenesis assays (40-43), no discernable difference in the extent of mesenchymal cell death was detected (reference control: wt GF mice injected with control IgG antibody; n=6 mice per group). In addition, administration of well-characterized neutralizing antibodies against Fgf-2, at a dose known to affect postnatal coronary angiogenesis in vivo (41), did not affect (enhance) the levels of mesenchymal cell death in GF wt mice compared to animals treated with an analogous Ig control (n=4 mice per group, data not shown). Together, these latter findings suggest that Vegf-A and Fgf2 are not essential for Fiaf-mediated radioprotection in GF mice.

### CONCLUSION

The ability to administer TBI, followed by transplantation of normal or genetically manipulated bone marrow to normal or genetically manipulated GF mice has revealed (i) the importance of the microbiota in conveying radiosensitivity to the small intestinal endothelium, (ii) the absence of a requirement for mature lymphocytes in conveying this microbiota-induced radiosensitive phenotype, and (iii) the contributions of Fiaf, a microbiota-regulated, epithelial-derived, secreted protein to radioresistance.

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.

### REFERENCES

1. Eckburg, P. B., Bik, E. M., Bernstein, C. N., Purdom, E., Dethlefsen, L., Sargent, M., Gill, S. R., Nelson, K. E. & Relman, D. A. (2005) Science. 308, 1635-1638
2. Backhed, F., Ley, R. E., Sonnenburg, J. L., Peterson, D. A. & Gordon, J. I. (2005) Science 307, 1915-20.
3. Backhed, F., Ding, H., Wang, T., Hooper, L. V., Koh, G. Y., Nagy, A., Semenkovich, C. F. & Gordon, J. I. (2004) Proc Natl Acad Sci USA 101, 15718-23.
4. Savage, D. C. (1986) Annu Rev Nutr 6, 155-78.
5. Hooper, L. V., Midtvedt, T. & Gordon, J. I. (2002) Annu Rev Nutr 22, 283-307.
6. Krul, C., Humblot, C., Philippe, C., Vermeulen, M., van Nuenen, M., Havenaar, R. & Rabot, S. (2002) Carcinogenesis 23, 1009-1016.
7. Humblot, C., Lhoste, E., Knasmuller, S., Gloux, K., Bruneau, A., Bensaada, M., Durao, J., Rabot, S., Andrieux, C. & Kassie, F. (2004) J Chromatography B 802, 231-237.
8. Abrams, G. D., Bauer, H. & Sprinz, H. (1963) Lab Invest 12, 355-64.
9. Lesher, S., Walburg, H. E., Jr. & Sacher, G. A., Jr. (1964) Nature 202, 884-6.
10. Khoury, K. A., Floch, M. H. & Hersh, T. (1969) J Exp Med 130, 659-70.
11. Savage, D. C., Siegel, J. E., Snellen, J. E. & Whitt, D. D. (1981) Appl Environ Microbiol 42, 996-1001.
12. Macpherson, A. J. & Uhr, T. (2004) Science 303, 1662-5.
13. Macdonald, T. T. & Monteleone, G. (2005) Science 307, 1920-5.
14. Stappenbeck, T. S., Hooper, L. V. & Gordon, J. I. (2002) Proc Nat Acad Sci USA 99, 15451-15455.
15. Somosy, Z., Horvath, G., Telbisz, A., Rez, G. & Palfia, Z. (2002) Micron 33, 167-178.
16. Livstone, E. M., Hersh, T., Spiro, H. M. & Floch, M. H. (1970) Yale J Biol Med 42, 448-54.
17. Brook, I., Walker, R. I. &, MacVittie, T. J. (1988) Int J Radiat Biol Relat Stud Phys Chem Med 53, 709-16.
18. Brook, I. & Ledney, G. D. (1992) Radiat Res 130, 61-4.
19. Reyniers, J. A., Trexler, P. C., Scruggs, W., Wagner, M. & Gordon, H. A. (1956) Radiation Res 5, 591.
20. McLaughlin, M. M., Dacquisto, M. P., Jacobus, D. P. & Horowitz, R. E. (1964) Radiat Res 23, 333-49.
21. Matsuzawa, T. (1965) Tohoku J. Exp. Med 85, 257-263.
22. Onoue, M., Uchida, K., Yokokura, T., Takahashi, T. & Mutai, M. (1981) Radiat Res 88, 533-41.
23. Wilson, R. (1968) J Am Osteopath Assoc 67, 653-62.
24. Heit, H., Fliedner, T. M., Fache, I. & Schnell, G. (1970) Radiat Res 41, 163-82.
25. Hooper, L. V., Mills, J. C., Roth, K. A., Stappenbeck, T. S., Wong, M. H. & Gordon, J. I. (2002) in Molecular Cellular Microbiology, eds. Sansonetti, P. & Zychlinsky, A. (Academic, San Diego), Vol. 31, pp. 559-589.
26. Motoike, T., Loughna, S., Perens, E., Roman, B. L., Liao, W., Chau, T. C., Richardson, C. D., Kawate, T., Kuno, J., Weinstein, B. M., Stainier, D. Y. & Sato, T. N. (2000) Genesis 28, 75-81.
27. Hanson, P., Mathews, V., Marrus, S. H. & Graubert, T. A. (2003) Exp Hematol 31, 159-167.
28. Paris, F., Fuks, Z., Kang, A., Capodieci, P., Juan, G., Ehleiter, D., Haimovitz-Friedman, A., Cordon-Cardo, C. & Kolesnick, R. (2001) Science 293, 293-297.
29. Ch'ang, H.-J., Maj, J. G., Paris, F., Xing, H. R., Zhang, J., Truman, J,-P., Cardon-Cardo, C., Haimovitz-Friedman, A., Kolesnick, R. & Fuks, Z. (2005) Nat Med 11, 484-490.
30. Cho, C.-H., Kammerer, R. A., Lee, H. J., Yasunaga, K., Kim, K.-T., Choi, H.-H., Kim, W., Kim, S.-H., Park, S. K., Lee, G.-M. & Koh, G.-Y. (2004) Proc Nat Acad Sci USA 101, 5553-5558.
31. Kim, I., Kim, H. G., Kim, H., Kim, H. H., Park, S. K., Uhm, C. S., Lee, Z. H. & Koh, G. Y. (2000) Biochem J 346 Pt 3, 603-10.
32. Mandard, S., Zandbergen, F., Tan, N. S., Escher, P., Patsouris, D., Koenig, W., Kleemann, R., Bakker, A., Veenman, F., Wahli, W., Muller, M. & Kersten, S. (2004) J Biol Chem 279, 34411-20.
33. Ge, H., Yang, G., Yu, X., Pourbahrami, T. & Li, C. (2004) J Lipid Res 45, 2071-9.
34. Ge, H., Yang, G., Huang, L., Motola, D. L., Pourbahrami, T. & Li, C. (2004) J Biol Chem 279, 2038-45.
35. Rawls, J, F., Samuel, B. S. & Gordon, J. I. (2004) Proc Natl Acad Sci USA 101, 4596-601.
36. Le Jan, S., Amy, C., Cazes, A., Monnot, C., Lamande, N., Favier, J., Philippe, J., Sibony, M., Gasc, J. M., Corvol, P. & Germain, S. (2003) Am J Pathol 162, 1521-8.
37. Chung, H. T., Pae, H. O., Choi, B. M., Billiar, T. R. & Kim, Y. M. (2001) Biochem Biophys Res Commun 282, 1075-9.
38. Walford, G. A., Moussignac, R. L., Scribner, A. W., Loscalzo, J. & Leopold, J. A. (2004) J Biol Chem 279, 4425-32.
39. Okunieff, P., Mester, M., Wang, J., Maddox, T., Gong, X., Tang, D., Coffee, M. & Ding, I. (1998) Radiat Res 150, 204-11.
40. Gorski, D. H., Beckett, M. A., Jaskowiak, N. T., Calvin, D. P., Mauceri, H. J., Salloum, R. M., Seetharam, S., Koons, A., Hari, D. M., Kufe, D. W. & Weichselbaum, R. R. (1999) Cancer Res 59, 3374-8.
41. Tomanek, R. J., Sandra, A., Zheng, W., Brock, T., Bjercke, R. J. & Holifield, J. S. (2001) Circ Res 88, 1135-41.
42. Nakayama, T., Yao, L. & Tosato, G. (2004) J Clin Invest 114, 1317-25.
43. Michel, F., Ambroisine, M. L., Duriez, M., Delcayre, C., Levy, B. I. & Silvestre, J. S. (2004) Circulation 109, 1933-7.
44. Stappenbeck, T. S., Hooper, L. V., Manchester, J. K., Wong, M. H. & Gordon, J. I. (2002) Methods Enzymol 356, 167-196.
45. Backhed, F., Ding, H., Wang, T., Hooper, L. V., Koh, G. Y., Nagy, A., Semenkovich, C. F.& Gordon, J. I. (2004) Proc Natl Acad Sci USA 101, 15718-23.
46. Gorski, D. H., Beckett, M. A., Jaskowiak, N. T., Calvin, D. P., Mauceri, H. J., Salloum, R. M., Seetharam, S., Koons, A., Hari, D. M., Kufe, D. W. & Weichselbaum, R. R. (1999) Cancer Res 59, 3374-8.
47. Tomanek, R. J., Sandra, A., Zheng, W., Brock, T., Bjercke, R. J. & Holifield, J. S. (2001) Circ Res 88, 1135-41.
48. Nakayama, T., Yao, L. & Tosato, G. (2004) J Clin Invest 114, 1317-25.
49. Michel, F., Ambroisine, M. L., Duriez, M., Delcayre, C., Levy, B. I. & Silvestre, J. S. (2004) Circulation 109, 1933-7.

1. A method for treating gastrointestinal radiation sensitivity in a subject, the method comprising administering to the subject an agent that increases either the amount or the activity of a Fiaf polypeptide in the subject.

2. The method of claim 1, wherein the subject has been diagnosed with cancer and is undergoing radiation therapy, the radiation therapy comprising exposing a part of the subject's body selected from the group consisting of the abdomen, pelvis, rectum, intestine, and total body to a radiation dose from about 5 Gy to about 90 Gy.

3. The method of claim 2, wherein the cancer is selected from the group consisting of rectal cancer, cancer of the anus, anal canal and anorectum cancer, peritoneum, omentum and mesentery cancer, cervical cancer, uterine cancer, ovarian cancer, colon cancer, cancer of the small intestine, prostate cancer, stomach cancer, cancer of the bladder, testicular cancer, kidney cancer, pancreatic cancer, liver cancer, vulvar cancer, cancer of the gallbladder, ureter cancer, and gastrointestinal carcinoid tumors.

4. The method of claim 1, wherein:
(a) the amount of Fiaf polypeptide is increased in the subject by administering to the subject an agent comprising a Fiaf polypeptide or a nucleotide sequence encoding a polypeptide having Fiaf activity; or
(b) the activity of the Fiaf polypeptide is increased by administering to the subject an agent comprising an agonist of a Fiaf polypeptide.

5. (canceled)

6. (canceled)

7. The method of claim 1, wherein the gastrointestinal radiation sensitivity comprises a disorder selected from the group consisting of diarrhea, dehydration, malabsorption, abdominal pain, rectal pain, proctitis, weight loss, nausea, vomiting, fatty stools, bloody stools, and frequent urges to have a bowel movement.

8. The method of claim 1, further comprising administering to the subject a second agent selected from the group consisting of an antidiarrheal agent, a rehydration therapy, a nutritional supplement, an analgesic, an antinausea agent, and an antispasmodic agent.

9. The method of claim 2, wherein the agent and the radiation are administered to the subject substantially simultaneously or sequentially.

10. (canceled)

11. A method for reducing the incidence of cellular apoptosis in the gastrointestinal tract of a subject, the method comprising administering to the subject an agent that increases either the amount or the activity of a Fiaf polypeptide in the subject.

12. The method of claim 11, wherein the subject has been diagnosed with cancer and is undergoing radiation therapy, the radiation therapy comprising exposing a part of the subject's body selected from the group consisting of the abdomen, pelvis, rectum, intestine, and total body to a radiation dose from about 5 Gy to about 90 Gy.

13. (canceled)

14. (canceled)

15. (canceled)

16. (canceled)

17. The method of claim 11, wherein:
(a) the amount of Fiaf polypeptide is increased in the subject by administering to the subject an agent comprising a Fiaf polypeptide or a nucleotide sequence encoding a polypeptide having Fiaf activity; or
(b) the activity of the Fiaf polypeptide is increased by administering to the subject an agent comprising an agonist of a Fiaf polypeptide.

18. (canceled)

19. The method of claim 12, wherein the agent and the radiation are administered to the subject substantially simultaneously or sequentially.

20. (canceled)

21. The method of claim 11, wherein the cell is an endothelial cell or a lymphocyte.

22. A combination for decreasing gastrointestinal radiosensitivity in a subject, the combination comprising a first agent that increases either the amount or the activity of a Fiaf polypeptide and a second agent that ameliorates at least one symptom of gastrointestinal radiosensitivity.

23. The combination of claim 22, wherein the first agent is a Fiaf polypeptide or a nucleotide sequence encoding a polypeptide having Fiaf activity.

24. The combination of claim 22, wherein the second agent is selected from the group consisting of an antidiarrheal agent, a rehydration therapy, a nutritional supplement, an analgesic, an antinausea agent, and an antispasmodic agent.

25. (canceled)

26. The combination of claim 22, wherein the subject has been diagnosed with cancer and is undergoing radiation therapy, the radiation therapy comprising exposing a part of the subject's body selected from the group consisting of the abdomen, pelvis, rectum, intestine, and total body to a radiation dose from about 5 Gy to about 90 Gy.

27. The combination of claim 26, wherein the cancer is selected from the group consisting of rectal cancer, cancer of the anus, anal canal and anorectum cancer, peritoneum, omentum and mesentery cancer, cervical cancer, uterine cancer, ovarian cancer, colon cancer, cancer of the small intestine, prostate cancer, stomach cancer, cancer of the bladder, testicular cancer, kidney cancer, pancreatic cancer, liver cancer, vulvar cancer, cancer of the gallbladder, ureter cancer, and gastrointestinal carcinoid tumors.

28. The combination of claim 26, further comprising a third agent selected from group consisting of a chemotherapeutic agent, fibroblast growth factor-2, vascular endothelial growth factor, and an inhibitor of acid sphingomyelinase.

29. (canceled)

30. The combination of claim 22, wherein the gastrointestinal radiation sensitivity comprises a disorder selected from the group consisting of diarrhea, dehydration, malabsorption, abdominal pain, rectal pain, proctitis, weight loss, nausea, vomiting, fatty stools, bloody stools, and frequent urges to have a bowel movement.

31. The composition of claim 28, wherein the first agent, the second agent and the radiation are administered to the subject substantially simultaneously or sequentially.

32. (canceled)


SEQUENCE LISTING (34 sequences)

SEQ ID NO: 1 - DNA, 1967 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 2 - DNA, 1843 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacactt

SEQ ID NO: 3 - DNA, 1853 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 4 - DNA, 1858 bp, Mus musculus
gcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgctcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctggacaatggagtagacaagacttcgaggggaaagaagctttccaagatgacccagctcattggcttgacttccaacgccacccacttacacaggccggcccgggactgccaggaactcttccaagaaggggagaggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacaggggaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgttgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacaaaacaaaaaaaaaaaaaaaaaaaa

SEQ ID NO: 5 - DNA, 1870 bp, Mus musculus
ttctgcaccagagcaagtctaagtctgagccggctcccccagaactccagctactgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtggacagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacaggggaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaaactagctgggcccttaatggcggactcagtcatattgactgactggagacagggtgccaggagccctggatacactcatggtgcttttttaggtgctgtggatgcacaggtgctaactgtggttcccaggcacagctcacagcattcttacaataaaaacaacctcagaaccaaaaaaaaaaaaaaaaaaaaaaaaaaaaaaa

SEQ ID NO: 6 - DNA, 1871 bp, Rattus norvegicus
cagctgcgatctacaggctttcttctgcaccagagcaagtctaagtctgagccggcttccccagaactccagctactgggtcttgaactcctgcgtcccgaagtcctagcgttgctgcacccaaggccatccccggaatcatgcgctgcgctccgaccgcaggcgctgctctagtgctatgcgcagctactgcggggctgctgagcgcgcaagggcgccctgcacagccggagccgccgcgcttcgcatcctgggatgaaatgaacttgctggctcacgggctgctgcagctcggtcacgggctgcgggaacacgtggagcgcacccgtggacagctgggcgcgctggaacgccgcatggctgcctgcggtaacgcttgccaggggcccaaggggacagacccgaaggatagagtccccgaaggccaggctcctgagactctgcagagtttacagactcaactcaaggctcagaacagcaagatccagcaactgttccagaaggtagcccagcagcagagatacctatcaaagcagaatctgagaatacagaatcttcagagccagattgacctcttgacccccacacacctagacaatggggtagacaagacttcgaggggaaagaggcttcccaagatggcccagctcattggcttgactcccaacgccacccgcttacacaggcctccccgggactgccaggaactctttcaagaaggggagcggcacagtggacttttccagatccagcctctgggatctccaccatttttggtcaactgtgagatgacttcagatggaggctggacggtgattcagagacgcctgaacggctctgtggacttcaatcagtcttgggaagcctacaaagatggcttcggagatccccaaggcgagttctggctgggcctagagaagatgcacagcatcacaggggaccgaggaagccagttggctgtgcagctccaggactgggatggcaatgccaaattgctccaatttcctatccatttggggggtgaggacacagcctacagcctgcagctcaccgagcccacggccaatgagctgggtgccaccaatgtttcccccaatggcctttccctgcccttctctacctgggaccaagaccacgacctccgaggggaccttaactgtgccaagagcctctctggtggctggtggtttggcacctgcagccattccaatctaaatggacaatacttccactctattccacggcaacggcagcagcgtaaaaaggggatcttctggaaaacatggaagggccgctactatccactacaggctaccaccctgttgatccagcccatggaggctacagcagcctcctagcctcctcactggagcctggttccaggcctaagaagacagtgactttggttgtgaccttaagatatggctgttttctgctgagagcaggagctctaagtagggctatctggagtctcgtggacagagaagaagcccataactggagtgactggaggaccccttttccgtgttggggtctgcaagcactgttgtctgaaacagtcagagcaacaggaaacaaatggcccagacccagaaaacgtgggctcaaggggcattgactctcacttctcgcctaccagagaagttggagatgcagagggaccatttcagtccaactagctgggcccttaatggcggactcggtcatattgaccgactggagatagggttcccaggagccctggatacactcaatggtgctcttgtgggtgctgtggatgaacaggtgccaactgtggttccaaggcacagctcacaacattcttacaataaaaacaacctcagaaaaaaaaaaaaaaaaaaaaaaaaaaa

SEQ ID NO: 7 - DNA, 1871 bp, Rattus norvegicus; identical to SEQ ID NO: 6

SEQ ID NO: 8 - DNA, 562 bp, Sus scrofa
  misc_feature (545)..(545): n is a, c, g, or t
gggtctccgccatttttggttaactgcaagatgacctcagatggaggctggactgtaattcagaggcgccaggatggctcggtggactttaaccagccttgggaagcctacaaggatggcttcggagatcccaaaggtgaattctggctgggcctggagaaggtgcaccgcatcatgggggaccgtggcagccgcctggctgtgcagctgcaagactgggagggcaatgccgagtccctgcagttccccgtccacctgggtggtgaggacactgcctacagcctgcagctcacggcacctgtggctagcaaactgggtgccaccatcgacacacccagtggcctctccctgcccttctccacttgggatcaggaccacgacctccgaggagacaagaactgcgccaagattctctctggtggttggtggtttggcacctgcagccactccaacctcaatggccagtacttccattccatccctcggcagcgggagcagcgtaagaagggcatcttctggaagacatggcaggaatcgaattcccngccgccatgcgccggac

SEQ ID NO: 9 - DNA, 1833 bp, Sus scrofa
ccctcacatcacgacctctgagtcctccttgcgccccctcgccccagcgtcccgaaatcctcgcgtcccagcctccggaccacctggaaggatgcgctctgcgccgacagccagagcagcgctggtgctgtgcgccgccaccgctgggctgctaagcgcgcagggcagcccggaacctcctgaggcgccgcgcttcgcgtcctgggacgaggtgaatgtgctggcgcacgggctcctgcagctcggccgcgggctgcgggagcacgtggagcgcacccgtgggcaactaggagcgctggaacggcgtctgagcgcgtgcggggccgcctgcaaagacccggaggggtccgccgtgcctcctctcaccgcggggaatctggtcccttcccaaagcgacgccgccccggagaccctccacagcttgcagacacaactcaaggctcagaacagcaagattcagcaactcttccagaaggtggcccagcagcagcggcacctggagaagcagcacttgagaatccagaatttgcagggccagctggaccacttggctcccatgcacctgggccatggggtggccaaggctgccaggagaaaaagactacccaagatgacccagcctgctggcccagctcacaatatcagccgcctgcacagactgcccagagactgccaagagctgtttgaagagggagagcggcaaagtggattgttccagatccagccccaggggtccccgccattcctggttaactgcaagatgacctcagatggaggctggactgtaattcagaggcgccaggatggctcggtggactttaaccagccttgggaagcctacaaggatggcttcggagatcccaaaggtgaattctggctgggcctggagaaggtgcaccgcatcatgggggaccgtggcagccgcctggctgtgcagctgcaagactgggagggcaatgccgagtccctgcagttccccgtccacctgggtggtgaggacactgcctacagcctgcagctcacggcacctgtggctagcaaactgggtgccaccatcgacacacccagtggcctctccctgcccttctccacttgggatcaggaccacgacctccgaggagacaagaactgcgccaagattccctctggtggttggtggtttggcacctgcagccactccaacctcaatggccagtacttccattccatccctcggcagcgggagcagcgtaagaagggcatcttttggaagacctggcggggccgctattacccgctgcaggccaccaccatgctgatccagcccacagtagccgaggtagcctcttagcctcctcgctggggcctggtcccaggctcccagaggacactgactctgactctggcccagcatgtggccactccttgcctgggcagggactccaggcaggagccagctggagcctcatggacagcgaagatgtccacgactggagaggcctttgatgagtggggggctgcaggaaacaccctccaggaaagatggagctgcagggctacagggcacagaccctgaaagcataggatggcgaggcacggaggcacactcttcacctgcctgaggagtgggggacacagagggaccactcggggacagccaggcaggccctcgatggcagattcagtcacattgactggctgggggaccaggacttctgtagggcccaggcaccctcagggtgctgttgtatgtgggtgctgtgagcaagccggcgccagtgccaacggcatccggggcaaaactcacagaattcttggaataaaagcaacctcagaacaaaaaaaaaaaaaaaa

SEQ ID NO: 10 - DNA, 271 bp, Bos taurus
tgctgcgaatccagaatctacagagccaggtgccagtgcttgctccagatggggagggaggttcaaggtggaccctgtgggtctggctgtggagccaggcaggttgagcagcctgaaccagccagacctgacatcctcctcctggtcctgtcctagatggatcacctggcccccaggcacctgggccacgagatggccaagcccgccaggaggaagaggctgcccaagatggcccagcttgctggcccagctcacaatatcagccgcctga

SEQ ID NO: 11 - DNA, 1970 bp, Bos taurus
cgcacagacacaccaagccgagggccgggggctgtcactctagtctttcctccggcttctgcaccgaagcgagtcctccgccagggccttcgggtttccagccctcgcacctcgaccccagagtctctctggcgccccctcattcctgcctctccgaatctccgcgttccgaagcctggaccaccaggaaggatgcgctgtgcgccgacagccggagcagccctgatgctgtgcgccgccaccgccgggctgctgagcgctcagggccgcccggagcctcccgagacgccgcgctttgcttcctgggacgaggtgaacgtgctggcacacggcctcttgcagctcggccacgggctgcgcgaacacgtggagcgcacccgtgggcagctgggcgagctggaacggcgtctgggcgcgtgtggggccgcctgcaaggatcctgaggggtccgccgcgcctccgcgtgcccaggccaatttggtcaatcccggcggcggcgacgcctccccagaaaccctccgcagcttgaagactcagctggaggctcagaacagcaggatccagcaactcttccagaaggtggcccagcagcagcggcacttggagaagcagcagctgcgaatccagaatctacagagccagatggatcacctggcccccaggcacctgggccacgagatggccaagcccgccaggaggaagaggctgcccaagatggcccagcttgctggcccagctcacaatatcagccgcctgcacagactgcccagggactgccaggagctgtttgaagagggagagagggaaagtggattgttccagatccagccccagggatccccgcctttcctggttaactgcaaaatgacctcagatggaggctggaccgtaattcagaggcgtcaggatggctccgtggactttaaccagccctgggaagcctacaaagatggcttcggagacccccaaggcgagttctggctgggcctggagaaggtgcatcacatccttggggaccgtggcagccgcctggctgtgcagctgcaggactgggagggcaatgccgagtccttgcagttccccatccacctggggggtgaagacaccgcctacagcctgcagctcacgccaccggtggccagcaaactgggcgccaccaccttctcgcccagtggcctctccctgcccttctccacttgggaccaggaccacgacctccgcggagacaagaactgcgccaggagcctctctggtggctggtggtttggcacctgcagtcactccaacctgaacggccagtatttccactccattccaaggcagcggcagcagcgtaagaagggcatcttctggaagacctggcggggccgctactacccactgcaggccaccaccatcctggtacagcccacagcggcctcctagcctcctctctggggcctggtttcaggcccccagaggacagtgactcttgtccacgtgtggccactccctgcctgggcaggggctccagacaagggctgtctggagcctcaaggacagagaagatgcccatgactggagaggtccccttgatgagtaggggaggctgcaggaaatgccctctgaggagagcagggctgtagggctgctgggcacagaccccgaaaacgtgggacagaggggcacagaaacccccctcacccgccaaggagcaggggatgcagaggggaccctgcgggggtggggcagccaggccggccctcgatggccagtggattcagtcacattgactggctggaggaccagggcttcccgtaggcctgggcaccctcaccgtactgtggtgcctgggtgctgtgagtcagctggcgcccacggcgtctgggtggaacccacagaattctcggaataaaagcaacttcaaaacaaaaaaaaaaaaaaaaaaaaaaaaaaaaaaaaaaaaaaaaaaaaaaaaaaaaaaaaaaaaa

SEQ ID NO: 12 - DNA, 1219 bp, Pan troglodytes
  misc_feature (240)..(247): n is a, c, g, or t
atgagcggtgctccgacggccggggcagccctgatgctctgcgccgccaccgccgtgctactgagcgctcagggcggccccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgnnnnnnnnccgaggggtccaccgacctcccgtnnnnccctgagagccgggtggaccctnnnnnncttcacagcctgcanacacaactcaaggcncagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccnagatggcccagccnnnngacccggctcacaatgtcagccgcctgcaccggctgcccagggattgccaggagctgttccaggttggggagaggcagagtggacnatttgaaatccagcctcaggggnnnccgccatttttggtnaactgcaagatgacctcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcgagttctggctgggtctggagaaggtgcatagcatcacgggggaccgcaacagccgcctggccgtgcagctgcgggactgggatggcaacgccgagtcgctgcagttctccgtgcacctgggtggcgaggacacggcctacagcctgcagctcactgcacccgtggccggccagctgggcgccaccaccatcccacccagcggcctctccgtanncttctccacttgggnnnnnnnnnnnnnnnnnnnnnnnnnnnnnnnnnnnnnnnnnnnnnnnnnnngaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcct

SEQ ID NO: 13 - DNA, 1726 bp, Macaca mulatta
atgtcctttgaagatctgatgggcatcccccagcccgcagactacctggcctccagaggacaggacccctcccccggccacaggccctgcccccgccactccccgcaccccgcctccaaggctcctccgcccactccgcacccaacttataaacacggtcctcgagcgcagccgggagaaacagagctaagcagatccacccctgtgatccgattctttccagcgccttctgcaaccaagcgggtatccccttggtcctccgcgtctccaaccctcgcacctggaactccagcgtccccgagagtccccgcatccccgctcccaggctacgtgagaggatgcgcggtgctccgacggccggagcagccctgatgctctgcgtcgccacggccgtgctgctgagagctcagggcggcccggtgcagtccaagtctccgcgcttcgcgtcctgggacgagatgaatgtcctggcgcacggactcctgcagctaggccaggggctgcgcgaacacgcggagcgcacccgcagtcagctgaacgcgctggagcggcgcctcagcgcttgcgggtctgcctgccagggaaccgaggggtccaccgccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcgtctgcaaagccaggttggcctcctggaccccaagcacctagaccatgaggtggccaagcctgcccgaagaaagaggcggcccgagatggcccagccagttgactcggctcacaatgccagccgcctgcaccggctgcccagggattgccaggagctgtttgaagatggggagaggcagagtggactatttgagatccagcctcaggggtctccgccatttttggtgaactgcaagatgacctcagccccattggtggctcaaagacctgaccatgtttcctctcccctgaccccggcaggaggctggtggtttggcacctgcagccattccaacctcaatggccagtacttccgctccattccacagcagcggcaggagcttaagaaaggaatcttctggaagacttggcggggccgctactacccgctgcaggccaccaccatgttgatccagcccacggcggcagaggcagcctcctagcctcctggttgggcctggtcccaggcccacgaaggacggtgactcttggctctgcccgaggatgtggccactccctgcccgggcaggggctccgaggaggggccatctggaagcttgtggacggagaagaagaccatggctggagaagccccctttctgagtgcaggggggctacatgcattgcctcctgagatcgaggctacaggatatgctcagactctggaggcgtggaccaaggggcatggagcttcactccttgctggctggggagctggggactcagagggactacttgaggctggccaggctggcctcagtggcggactcagtcacattgactgactgggggaccagggctcacgtgagtcaagaacgccctcctggtgcgggtgctgttgtgtgtaggtcccctggggacacaagtaggcaccagtcgtgtctgggcggagctcacagagttcttggaataaaagcaacctcagaacacttcg

SEQ ID NO: 14 - DNA, 2053 bp, Canis familiaris
atggggataggagcgtcttacccaccgctacggtcccagcacagtagctgtcacacagggtaccgccagcagcgtcagtccccgccgagcgcggcgggcgcggccctggtgctgtacgccgccgccgccggcttcctgattgcgcggggcagcccgacgccccccgagccgcagcgcttcgcgtcctgggacgaggtgaacgtgctggcgcacgggctcctgcagctcggccacgggctgcgcgagcacgtggagcgcacccaggggcagctgggcgccctggagcggcgtctgggcgcctgcggggcagcctgcaaggagcctgggggctccgccgcgcctccgcgcgcccccgagggcttgggcccccgcggcgaggctgcccccgaggctctccgcagcctgcagactcagctcaagacccagaacagcaggatcgagcaactcttccacaaggtggcccagcagcagcggcacctggagaagcagcagctgaaaatacagaagctgcagagccaggtgggcctcctgggccccatgcatctgggccatgggatggtgaaggctgccaggaagaagaggctccccaagatggcacagctcgtgggccctgctcacaacatcagccgcctgcaccggctgcccagggactgccaagagctgtttgaagaaggagagaggcaaagtggactgttccagatccagcctcaggggtccccgcccttcctggttaactgcaagatgaccgcagatggaggctggactgtcattcagagacgccaggatggctccgtggactttaatcagccctgggaagcctacaaggctggctttggagacccgcacggtgagttctggctgggcctggagaaggtgcaccgcatcctgggagaccgcggcagccgcctggctgtgcagcttcaggactgggagggcaatgcggagtccctgcagttccccgtccatctgggtggtgaggacacggcctacagcctgcagctcacggcgcctgtggccaatgaactgggggccaccactgtggcgcccagtggcctttccctgcccttctcgacttgggatcaggaccacgacctgcgtggtgacaagaactgtgccaagagcctctctggtggctggtggtttggcacctgtggccattccaacctcaacggccagtacttccgttccattccacaccagcggcagcagcgtaagaagggcatcttctggaagacctggcggggccgctactacccactgcaggccaccacgatgctgatccagcccacagcaactgaggcagcctcctagcttcctcactggggcctggtgccaggctcctggaggacagtgactctgactctggcccggcatgtggccactccctgcccaggcaggggctcaagacaggggccctctggagcctcgtggacagagaagcagctcatgactggagttgccccctttccgggtggggggctgcaggtgataccctctgaggacggagctgcagggttgcaggccctggaagcgtgagaccgaggggctttgagacctattccttgcctgcccgaggagtcggacgtagagggaccatttgggggcagccaggccagccctcgatggaggattcagtcacattgactggctgggggaccagggctcccgtgggcccccgcgggcccccagcgccctcacagtgtccttgtgtgtgggtgctgtgggatgagccggcgccagcggtgtccgggtggagctcgcagaattcttgtaataaaagcaacctcagaacattttgttctgttttattcttcagtttgttttaaaaagtgggcagtttcaaagtccacagaaatacgttcccagggtggagggtgaaggctcccagatcctttgaggacgacactggctcgtcctaccacccgacaacaggttttaatttcctcccttcgtttgttgtttcacaatgtgtgtgtcaggttttatattcaaaaactgaaatacttaatccttctggaaactcgtggagagaataaaagccccttcttaatta

SEQ ID NO: 15 - protein, 368 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHHGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 16 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 17 - protein, 406 aa, Homo sapiens; identical to SEQ ID NO: 16

SEQ ID NO: 18 - protein, 410 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGDRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 19 - protein, 410 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 20 - protein, 410 aa, Bos taurus
MRCAPTAGAALMLCAATAGLLSAQGRPEPPETPRFASWDEVNVLAHGLLQLGHGLREHVERTRGQLGELERRLGACGAACKDPEGSAAPPRAQANLVNPGGGDASPETLRSLKTQLEAQNSRIQQLFQKVAQQQRHLEKQQLRIQNLQSQMDHLAPRHLGHEMAKPARRKRLPKMAQLAGPAHNISRLHRLPRDCQELFEEGERESGLFQIQPQGSPPFLVNCKMTSDGGWTVIQRRQDGSVDFNQPWEAYKDGFGDPQGEFWLGLEKVHHILGDRGSRLAVQLQDWEGNAESLQFPIHLGGEDTAYSLQLTPPVASKLGATTFSPSGLSLPFSTWDQDHDLRGDKNCARSLSGGWWFGTCSHSNLNGQYFHSIPRQRQQRKKGIFWKTWRGRYYPLQATTILVQPTAAS

SEQ ID NO: 21 - protein, 459 aa, Danio rerio
MKVPLANLLCITVLASSGTSFPMERRGAAAGKEKRVQYAAWDDVNVLAHGMLQLGQGLKEHVDKTKGQVRDITIKMKVFNVTVSELGKLTQQLQEDNELLKAKAQNLEDSESLVLNVSTDLRQKTDELLKDRQKDHERMNKLEEKVDGLMQGEGLEAANSNYSDARIIQWMLEAQNKRIDDLVERIKQQQEKLDKQNIRIRTLQNQITMKNERLSLKRMEEDVNLNASTEQRDSPVALASDCHELFLRGETSSGLYTIQPSDSQPFEVYCEMTPGGWTVIQRRQDGSVDFDQLWQAYQNGFGNLNGEFWLGLEKIHSVSKGGNYILKVQFSDWRDEIQSISYRFHLNGQENNYSLRILESPAGNTESSLPTETSAVPFSTRDKDNDQKNDLNCAKQLSGGWWFSNCGRSNLNGRYFVTPAPKQRHQRKQGVFWKTWRGRYYPLKTTTMMIAPAEIVNKS

SEQ ID NO: 22 - DNA, 21 bp, Artificial Sequence
  mus musculus
cagctgggctgtacaaacctt

SEQ ID NO: 23 - DNA, 21 bp, Artificial Sequence
  mus musculus
cattggaagtgaagcgtttcg

SEQ ID NO: 24 - DNA, 24 bp, Artificial Sequence
  mus musculus
agtcccatgaagtgatcaagttca

SEQ ID NO: 25 - DNA, 19 bp, Artificial Sequence
  mus musculus
atccgcatgatctgcatgg

SEQ ID NO: 26 - DNA, 23 bp, Artificial Sequence
  mus musculus
gtcacggaaatactccagttggt

SEQ ID NO: 27 - DNA, 20 bp, Artificial Sequence
  mus musculus
cccgttttggatccgagttt

SEQ ID NO: 28 - DNA, 22 bp, Artificial Sequence
  mus musculus
agagcactcctgtcaatggctt

SEQ ID NO: 29 - DNA, 22 bp, Artificial Sequence
  mus musculus
tgtgaagggcatcagctggtaa

SEQ ID NO: 30 - DNA, 21 bp, Artificial Sequence
  mus musculus
caatgccaaattgctccaatt

SEQ ID NO: 31 - DNA, 16 bp, Artificial Sequence
  mus musculus
tggccgtgggctcagt

SEQ ID NO: 32 - DNA, 21 bp, Artificial Sequence
  mus musculus
cctctggtgaagcccaagatc

SEQ ID NO: 33 - DNA, 19 bp, Artificial Sequence
  mus musculus
tctgggtttccgccagttt

SEQ ID NO: 34 - protein, 405 aa, Rattus norvegicus
MRCAPTAGAALVLCAATAGLLSAQGRPAQPEPPRFASWDEMNLLAHGLLQLGHGLREHVERTRGQLGALERRMAACGNACQGPKGTDPKDRVPEGQAPETLQSLQTQLKAQNSKIQQLFQKVAQQQRYLSKQNLRIQNLQSQIDLLTPTHLDNGVDKTSRGKRLPKMAQLIGLTPNATRLHRPPRDCQELFQEGERHSGLFQIQPLGSPPFLVNCEMTSDGGWTVIQRRLNGSVDFNQSWEAYKDGFGDPQGEFWLGLEKMHSITGDRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQQRKKGIFWKTWKGRYYPLQATTLLIQPMEATAAS